(19) United States
(12) Patent Application Publication
(10) Pub. No.: US 2025/0163414 A1
(43) Pub. Date: May 22, 2025

(54) ANTISENSE OLIGONUCLEOTIDES FOR INDUCING EXON SKIPPING AND METHODS OF USE THEREOF

(71) Applicant: The University of Western Australia, Crawley (AU)

(72) Inventors: Stephen Donald WILTON, Applecross (AU); Sue FLETCHER, Bayswater (AU); Graham MCCLOREY, Bayswater (AU)

(73) Assignee: The University of Western Australia, Crawley (AU)

(21) Appl. No.: 18/785,527

(22) Filed: Jul. 26, 2024

Related U.S. Application Data
(63) Continuation of application No. 18/525,404, filed on Nov. 30, 2023, now abandoned.
Application No. 18/525,404 is a continuation of application No. 18/166,239, filed on Feb. 8, 2023, now abandoned.
Application No. 18/166,239 is a continuation of application No. 17/854,733, filed on Jun. 30, 2022, now abandoned.
Application No. 17/854,733 is a continuation of application No. 16/997,022, filed on Aug. 19, 2020, now abandoned.
Application No. 16/997,022 is a continuation of application No. 16/458,929, filed on Jul. 1, 2019, now Pat. No. 10,781,451.
Application No. 16/458,929 is a continuation of application No. 15/673,019, filed on Aug. 9, 2017, now abandoned.
Application No. 15/673,019 is a continuation of application No. 15/388,532, filed on Dec. 22, 2016, now abandoned.
Application No. 15/388,532 is a continuation of application No. 15/164,090, filed on May 25, 2016, now abandoned.
Application No. 15/164,090 is a continuation of application No. 14/883,070, filed on Oct. 14, 2015, now abandoned.
Application No. 14/883,070 is a continuation of application No. 14/223,634, filed on Mar. 24, 2014, now abandoned.
Application No. 14/223,634 is a continuation of application No. 13/963,578, filed on Aug. 9, 2013, now abandoned.
Application No. 13/963,578 is a continuation of application No. 13/727,415, filed on Dec. 26, 2012, now abandoned.
Application No. 13/727,415 is a continuation of application No. 12/837,356, filed on Jul. 15, 2010, now Pat. No. 8,524,880.
Application No. 12/837,356 is a continuation of application No. 11/570,691, filed on Jan. 15, 2008, now Pat. No. 7,807,816.

(30) Foreign Application Priority Data
Jun. 28, 2004 (AU) 2004903474

(51) Int. Cl. C12N 15/113

(52) U.S. Cl. CPC C12N 15/113; C12N 2310/11; C12N 2310/315; C12N 2310/321; C12N 2310/3233; C12N 2310/33; C12N 2310/3341; C12N 2310/3519; C12N 2320/30; C12N 2320/33

(57) ABSTRACT

An antisense molecule capable of binding to a selected target site to induce exon skipping in the dystrophin gene, as set forth in SEQ ID NO: 1 to 202.

### CROSS-REFERENCE TO RELATED APPLICATIONS

This application is a continuation of U.S. Patent Application No. 15/388,532, filed Dec. 22, 2016, pending, which application is a continuation of U.S. patent application Ser. No. 15/164,090, filed May 25, 2016, abandoned, which application is a continuation of U.S. patent application Ser. No. 14/883,070, filed Oct. 14, 2015, abandoned, which application is a continuation of U.S. patent application Ser. No. 14/223,634, filed Mar. 24, 2014, abandoned, which application is a continuation of U.S. patent application Ser. No. 13/963,578, Aug. 9, 2013, abandoned, which is a continuation of U.S. patent application Ser. No. 13/727,415, filed Dec. 26, 2012, abandoned, which application is a continuation of U.S. patent application Ser. No. 12/837,356, filed Jul. 15, 2010, now issued as U.S. Pat. No. 8,524,880, which application is a continuation of U.S. patent application Ser. No. 11/570,691, filed Jan. 15, 2008, now issued as U.S. Pat. No. 7,807,816, which application is a 35 U.S.C. §371 National Phase Application of PCT/AU2005/000943, filed Jun. 28, 2005, which claims priority to Australian Patent Application No. 2004903474, filed Jun. 28, 2004; which applications are each incorporated herein by reference in their entireties.

### STATEMENT REGARDING SEQUENCE LISTING

The Sequence Listing associated with this application has been submitted electronically in ASCII format, and is hereby incorporated by reference into the specification in its entirety. The name of the text file containing the Sequence Listing is Sequence-Listing_AVN-008CN40.txt. The text file is 62,133 Kilobytes, was created on Aug. 8, 2017 and is being submitted electronically via EFS-Web.

### FIELD OF THE INVENTION

The present invention relates to novel antisense compounds and compositions suitable for facilitating exon skipping. It also provides methods for inducing exon skipping using the novel antisense compounds as well as therapeutic compositions adapted for use in the methods of the invention.

### BACKGROUND ART

Significant effort is currently being expended researching methods for suppressing or compensating for disease-causing mutations in genes. Antisense technologies are being developed using a range of chemistries to affect gene expression at a variety of different levels (transcription, splicing, stability, translation). Much of that research has focused on the use of antisense compounds to correct or compensate for abnormal or disease-associated genes in a myriad of different conditions.

Antisense molecules are able to inhibit gene expression with exquisite specificity and because of this many research efforts concerning oligonucleotides as modulators of gene expression have focused on inhibiting the expression of targeted genes such as oncogenes or viral genes. The antisense oligonucleotides are directed either against RNA(sense strand) or against DNA where they form triplex structures inhibiting transcription by RNA polymerase II. To achieve a desired effect in specific gene down-regulation, the oligonucleotides must either promote the decay of the targeted mRNA or block translation of that mRNA, thereby effectively preventing de novo synthesis of the undesirable target protein.

Such techniques are not useful where the object is to up-regulate production of the native protein or compensate for mutations which induce premature termination of translation such as nonsense or frame-shifting mutations. Furthermore, in cases where a normally functional protein is prematurely terminated because of mutations therein, a means for restoring some functional protein production through antisense technology has been shown to be possible through intervention during the splicing processes (Sierakowska H, et al., (1996) Proc Natl Acad Sci USA 93, 12840-12844; Wilton S D, et al., (1999) Neuromusc Disorders 9, 330-338; van Deutekom J C et al., (2001) Human Mol Genet 10, 1547-1554). In these cases, the defective gene transcript should not be subjected to targeted degradation so the antisense oligonucleotide chemistry should not promote target mRNA decay.

In a variety of genetic diseases, the effects of mutations on the eventual expression of a gene can be modulated through a process of targeted exon skipping during the splicing process. The splicing process is directed by complex multi-particle machinery that brings adjacent exon-intron junctions in pre-mRNA into close proximity and performs cleavage of phosphodiester bonds at the ends of the introns with their subsequent reformation between exons that are to be spliced together. This complex and highly precise process is mediated by sequence motifs in the pre-mRNA that are relatively short semi-conserved RNA segments to which bind the various nuclear splicing factors that are then involved in the splicing reactions. By changing the way the splicing machinery reads or recognises the motifs involved in pre-mRNA processing, it is possible to create differentially spliced mRNA molecules. It has now been recognised that the majority of human genes are alternatively spliced during normal gene expression, although the mechanisms invoked have not been identified. Using antisense oligonucleotides, it has been shown that errors and deficiencies in a coded mRNA could be bypassed or removed from the mature gene transcripts.

In nature, the extent of genetic deletion or exon skipping in the splicing process is not fully understood, although many instances have been documented to occur, generally at very low levels (Sherrat T G, et al., (1993) Am J Hum Genet 53, 1007-1015). However, it is recognised that if exons associated with disease-causing mutations can be specifically deleted from some genes, a shortened protein product can sometimes be produced that has similar biological properties of the native protein or has sufficient biological activity to ameliorate the disease caused by mutations associated with the target exon (Lu Q L, et al., (2003) Nature Medicine 9, 1009-1014; Aartsma-Rus A et al., (2004) Am J Hum Genet 74:83-92).

This process of targeted exon skipping is likely to be particularly useful in long genes where there are many exons and introns, where there is redundancy in the genetic constitution of the exons or where a protein is able to function without one or more particular exons (e.g. with the dystrophin gene, which consists of 79 exons; or possibly some collagen genes which encode for repeated blocks of sequence or the huge nebulin or titin genes which are comprised of ˜80 and over 370 exons, respectively).

Efforts to redirect gene processing for the treatment of genetic diseases associated with truncations caused by mutations in various genes have focused on the use of antisense oligonucleotides that either: (1) fully or partially overlap with the elements involved in the splicing process; or (2) bind to the pre-mRNA at a position sufficiently close to the element to disrupt the binding and function of the splicing factors that would normally mediate a particular splicing reaction which occurs at that element (e.g., binds to the pre-mRNA at a position within 3, 6, or 9 nucleotides of the element to be blocked).

For example, modulation of mutant dystrophin pre-mRNA splicing with antisense oligoribonucleotides has been reported both in vitro and in vivo. In one type of dystrophin mutation reported in Japan, a 52-base pair deletion mutation causes exon 19 to be removed with the flanking introns during the splicing process (Matsuo et al., (1991) J Clin Invest., 87:2127-2131). An in vitro minigene splicing system has been used to show that a 31-mer 2′-O-methyl oligoribonucleotide complementary to the 5′ half of the deleted sequence in dystrophin Kobe exon 19 inhibited splicing of wild-type pre-mRNA (Takeshima et al. (1995), J. Clin. Invest. 95, 515-520). The same oligonucleotide was used to induce exon skipping from the native dystrophin gene transcript in human cultured lymphoblastoid cells.

Dunckley et al., (1997) Nucleosides & Nucleotides, 16, 1665-1668 described in vitro constructs for analysis of splicing around exon 23 of mutated dystrophin in the mdx mouse mutant, a model for muscular dystrophy. Plans to analyse these constructs in vitro using 2′ modified oligonucleotides targeted to splice sites within and adjacent to mouse dystrophin exon 23 were discussed, though no target sites or sequences were given.

2′-O-methyl oligoribonucleotides were subsequently reported to correct dystrophin deficiency in myoblasts from the mdx mouse from this group. An antisense oligonucleotide targeted to the 3′ splice site of murine dystrophin intron 22 was reported to cause skipping of the mutant exon as well as several flanking exons and created a novel in-frame dystrophin transcript with a novel internal deletion. This mutated dystrophin was expressed in 1-2% of antisense treated mdx myotubes. Use of other oligonucleotide modifications such as 2′-O-methoxyethyl phosphodiesters are described (Dunckley et al. (1998) Human Mol. Genetics, 5, 1083-90).

Thus, antisense molecules may provide a tool in the treatment of genetic disorders such as Duchenne Muscular Dystrophy (DMD). However, attempts to induce exon skipping using antisense molecules have had mixed success. Studies on dystrophin exon 19, where successful skipping of that exon from the dystrophin pre-mRNA was achieved using a variety of antisense molecules directed at the flanking splice sites or motifs within the exon involved in exon definition as described by Errington et al. (2003) J Gen Med 5, 518-527”.

In contrast to the apparent ease of exon 19 skipping, the first report of exon 23 skipping in the mdx mouse by Dunckley et al., (1998) is now considered to be reporting only a naturally occurring revertant transcript or artefact rather than any true antisense activity. In addition to not consistently generating transcripts missing exon 23, Dunckley et al., (1998) did not show any time course of induced exon skipping, or even titration of antisense oligonucleotides, to demonstrate dose dependent effects where the levels of exon skipping corresponded with increasing or decreasing amounts of antisense oligonucleotide. Furthermore, this work could not be replicated by other researchers.

The first example of specific and reproducible exon skipping in the mdx mouse model was reported by Wilton et al., (1999) Neuromuscular Disorders 9, 330-338. By directing an antisense molecule to the donor splice site, consistent and efficient exon 23 skipping was induced in the dystrophin mRNA within 6 hours of treatment of the cultured cells. Wilton et al, (1999), also describe targeting the acceptor region of the mouse dystrophin pre-mRNA with longer antisense oligonucleotides and being unable to repeat the published results of Dunckley et al., (1998). No exon skipping, either 23 alone or multiple removal of several flanking exons, could be reproducibly detected using a selection of antisense oligonucleotides directed at the acceptor splice site of intron 22.

While the first antisense oligonucleotide directed at the intron 23 donor splice site induced consistent exon skipping in primary cultured myoblasts, this compound was found to be much less efficient in immortalized cell cultures expressing higher levels of dystrophin. However, with refined targeting and antisense oligonucleotide design, the efficiency of specific exon removal was increased by almost an order of magnitude (see Mann C J et al., (2002) J Gen Med 4, 644-654).

Thus, there remains a need to provide antisense oligonucleotides capable of binding to and modifying the splicing of a target nucleotide sequence. Simply directing the antisense oligonucleotides to motifs presumed to be crucial for splicing is no guarantee of the efficacy of that compound in a therapeutic setting.

### SUMMARY OF THE INVENTION

The present invention provides antisense molecule compounds and compositions suitable for binding to RNA motifs involved in the splicing of pre-mRNA that are able to induce specific and efficient exon skipping and a method for their use thereof.

The choice of target selection plays a crucial role in the efficiency of exon skipping and hence its subsequent application of a potential therapy. Simply designing antisense molecules to target regions of pre-mRNA presumed to be involved in splicing is no guarantee of inducing efficient and specific exon skipping. The most obvious or readily defined targets for splicing intervention are the donor and acceptor splice sites although there are less defined or conserved motifs including exonic splicing enhancers, silencing elements and branch points.

The acceptor and donor splice sites have consensus sequences of about 16 and 8 bases respectively (see FIG. 1 for schematic representation of motifs and domains involved in exon recognition, intron removal and the splicing process).

According to a first aspect, the invention provides antisense molecules capable of binding to a selected target to induce exon skipping.

For example, to induce exon skipping in exons 3 to 8, 10 to 16, 19 to 40, 42 to 44, 46, 47, and 50 to 53 in the Dystrophin gene transcript the antisense molecules are preferably selected from the group listed in Table 1A.

In a further example, it is possible to combine two or more antisense oligonucleotides of the present invention together to induce multiple exon skipping in exons 19-20, and 53. This is a similar concept to targeting of a single exon. A combination or “cocktail” of antisense oligonucleotides are directed at adjacent exons to induce efficient exon skipping.

In another example, to induce exon skipping in exons 19-20, 31, 34 and 53 it is possible to improve exon skipping of a single exon by joining together two or more antisense oligonucleotide molecules. This concept is termed by the inventor as a “weasel”, an example of a cunningly designed antisense oligonucleotide. A similar concept has been described in Aartsma-Rus A et al., (2004) Am J Hum Genet 74:83-92).

According to a second aspect, the present invention provides antisense molecules selected and or adapted to aid in the prophylactic or therapeutic treatment of a genetic disorder comprising at least an antisense molecule in a form suitable for delivery to a patient.

According to a third aspect, the invention provides a method for treating a patient suffering from a genetic disease wherein there is a mutation in a gene encoding a particular protein and the affect of the mutation can be abrogated by exon skipping, comprising the steps of: (a) selecting an antisense molecule in accordance with the methods described herein; and (b) administering the molecule to a patient in need of such treatment.

The invention also addresses the use of purified and isolated antisense oligonucleotides of the invention, for the manufacture of a medicament for treatment of a genetic disease.

The invention further provides a method of treating a condition characterised by Duchenne muscular dystrophy, which method comprises administering to a patient in need of treatment an effective amount of an appropriately designed antisense oligonucleotide of the invention, relevant to the particular genetic lesion in that patient. Further, the invention provides a method for prophylactically treating a patient to prevent or at least minimise Duchene muscular dystrophy, comprising the step of: administering to the patient an effective amount of an antisense oligonucleotide or a pharmaceutical composition comprising one or more of these biological molecules.

The invention also provides kits for treating a genetic disease, which kits comprise at least a antisense oligonucleotide of the present invention, packaged in a suitable container and instructions for its use.

Other aspects and advantages of the invention will become apparent to those skilled in the art from a review of the ensuing description, which proceeds with reference to the following figures.

### BRIEF DESCRIPTION OF THE DRAWINGS

FIG. 1 Schematic representation of motifs and domains involved in exon recognition, intron removal and the splicing process (SEQ ID NOS: 213 and 214).

FIG. 2. Diagrammatic representation of the concept of antisense oligonucleotide induced exon skipping to by-pass disease-causing mutations (not drawn to scale). The hatched box represents an exon carrying a mutation that prevents the translation of the rest of the mRNA into a protein. The solid black bar represents an antisense oligonucleotide that prevents inclusion of that exon in the mature mRNA.

FIG. 3 Gel electrophoresis showing differing efficiencies of two antisense molecules directed at exon 8 acceptor splice site. The preferred compound [H8A(−06+18)] induces strong and consistent exon skipping at a transfection concentration of 20 nanomolar in cultured normal human muscle cells. The less preferred antisense oligonucleotide [H8A(−06+14)] also induces efficient exon skipping, but at much higher concentrations. Other antisense oligonucleotides directed at exon 8 either only induced lower levels of exon skipping or no detectable skipping at all (not shown).

FIG. 4 Gel electrophoresis showing differing efficiencies of two antisense molecules directed at internal domains within exon 7, presumably exon splicing enhancers. The preferred compound [H7A(+45+67)] induces strong and consistent exon skipping at a transfection concentration of 20 nanomolar in cultured human muscle cells. The less preferred antisense oligonucleotide [H7A(+2+26)] induces only low levels of exon skipping at the higher transfection concentrations. Other antisense oligonucleotides directed at exon 7 either only induced lower levels of exon skipping or no detectable skipping at all (not shown).

FIG. 5 Gel electrophoresis showing an example of low efficiency exon 6 skipping using two non-preferred antisense molecules directed at human exon 6 donor splice site. Levels of induced exon 6 skipping are either very low [H6D(+04−21)] or almost undetectable [H6D(+18−04)]. These are examples of non-preferred antisense oligonucleotides to demonstrate that antisense oligonucleotide design plays a crucial role in the efficacy of these compounds.

FIG. 6 Gel electrophoresis showing strong and efficient human exon 6 skipping using an antisense molecules [H6A(+69+91)] directed at an exon 6 internal domain, presumably an exon splicing enhancer. This preferred compound induces consistent exon skipping at a transfection concentration of 20 nanomolar in cultured human muscle cells.

FIG. 7 Gel electrophoresis showing strong human exon 4 skipping using an antisense molecule H4A(+13+32) directed at an exon 6 internal domain, presumably an exon splicing enhancer. This preferred compound induces strong and consistent exon skipping at a transfection concentration of 20 nanomolar in cultured human muscle cells,

FIG. 8A Gel electrophoresis showing strong human exon 12 skipping using antisense molecule H12A(+52+75) directed at exon 12 internal domain.

FIG. 8B Gel electrophoresis showing strong human exon 11 skipping using antisense molecule H11A(+75+97) directed at an exon 11 internal domain.

FIG. 9A Gel electrophoresis showing strong human exon 15 skipping using antisense molecules H15A(+48+71) and H15A(−12+19) directed at an exon 15 internal domain.

FIG. 9B Gel electrophoresis showing strong human exon 16 skipping using antisense molecules H16A(−12+19) and H16A(−06+25),

FIG. 10 Gel electrophoresis showing human exon 19/20 skipping using antisense molecules H20A(+44+71) and H20A(+149+170) directed at an exon 20 and a “cocktail” of antisense oligonucleotides H19A(+35+65, H20A(+44+71) and H20A(+149+170) directed at exons 19/20.

FIG. 11 Gel electrophoresis showing human exon 19/20 skipping using “weasels” directed at exons 19 and 20.

FIG. 12 Gel electrophoresis showing exon 22 skipping using antisense molecules H22A(+125+106), H22A(+47+69), H22A(+80+101) and H22D(+13−11) directed at exon 22.

FIG. 13 Gel electrophoresis showing exon 31 skipping using antisense molecules H31D(+01−25) and H31D(+03−22); and a “cocktail” of antisense molecules directed at exon 31.

FIG. 14 Gel electrophoresis showing exon 33 skipping using antisense molecules H33A(+30+56) and H33A(+64+88) directed at exon 33.

FIG. 15 Gel electrophoresis showing exon 35 skipping using antisense molecules H35A(+141+161), H35A(+116+135), and H35A(+24+43) and a “cocktail of two antisense molecules, directed at exon 35.

FIG. 16 Gel electrophoresis showing exon 36 skipping using antisense molecules H32A(+49+73) and H36A(+26+50) directed at exon 36.

FIG. 17 Gel electrophoresis showing exon 37 skipping using antisense molecules H37A(+82+105) and H37A(+134+157) directed at exon 37.

FIG. 18 Gel electrophoresis showing exon 38 skipping using antisense molecule H38A(+88+112) directed at exon 38.

FIG. 19 Gel electrophoresis showing exon 40 skipping using antisense molecule H40A(−05+17) directed at exon 40.

FIG. 20 Gel electrophoresis showing exon 42 skipping using antisense molecule H42A(−04+23) directed at exon 42.

FIG. 21 Gel electrophoresis showing exon 46 skipping using antisense molecule H46A(+86+115) directed a #exon 46

FIG. 22 Gel electrophoresis showing exon 51, exon 52 and exon 53 skipping using various antisense molecules directed at exons 51, 52 and 53, respectively. A “cocktail” of antisense molecules is also shown directed at exon 53.

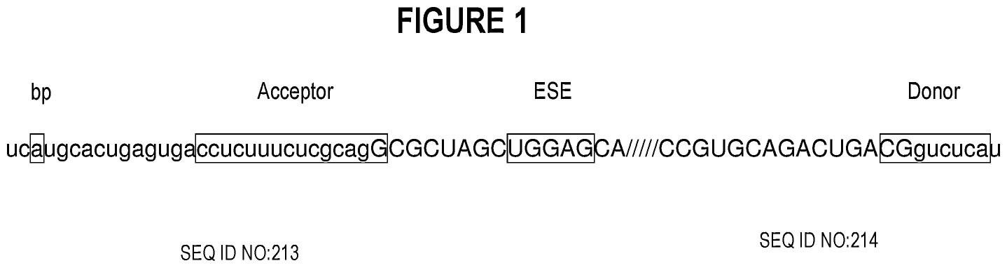

### BRIEF DESCRIPTION OF THE SEQUENCE LISTINGS

TABLE 1A
Description of 2′-O-methyl phosphorothioate antisense oligonucleotides that
have been used to date to study induced exon skipping during the processing of the
dystrophin pre-mRNA. Since these 2′-O-methyl antisense oligonucleotides are more RNA-
like, U represents uracil. With other antisense chemistries such as peptide nucleic acids or
morpholinos, these U bases may be shown as “T”.

| SEQ | | |
|---|---|---|
| ID | SEQUENCE | NUCLEOTIDE SEQUENCE (5′ - 3′) |
| 1 | H8A(−06 +18) | GAU AGG UGG UAU CAA CAU CUG UAA |
| 2 | H8A (−03 +18) | GAU AGG UGG UAU CAA CAU CUG |
| 3 | H8A(−07 +18) | GAU AGG UGG UAU CAA CAU CUG UAA G |
| 4 | H8A(−06 +14) | GGU GGU AUC AAC AUC UGU AA |
| 5 | H8A(−10 +10) | GUA UCA ACA UCU GUA AGC AC |
| 6 | H7A(+45 +67) | UGC AUG UUC CAG UCG UUG UGU GG |
| 7 | H7A(+02 +26) | CAC UAU UCC AGU CAA AUA GGU CUG G |
| 8 | H7D(+15 −10) | AUU UAC CAA CCU UCA GGA UCG AGU A |
| 9 | H7A(−18 +03) | GGC CUA AAA CAC AUA CAC AUA |
| 10 | C6A(−10 +10) | CAU UUU UGA CCU ACA UGU GG |
| 11 | C6A(−14 +06) | UUU GAC CUA CAU GUG GAA AG |
| 12 | C6A(−14 +12) | UAC AUU UUU GAC CUA CAU GUG GAA AG |
| 13 | C6A(−13 +09) | AUU UUU GAC CUA CAU GGG AAA G |
| 14 | CH6A(+69 +91) | UAC GAG UUG AUU GUC GGA CCC AG |
| 15 | C6D(+12 −13) | GUG GUC UCC UUA CCU AUG ACU GUG G |
| 16 | C6D(+06 −11) | GGU CUC CUU ACC UAU GA |
| 17 | H6D(+04 −21) | UGU CUC AGU AAU CUU CUU ACC UAU |
| 18 | H6D(+18 −04) | UCU UAC CUA UGA CUA UGG AUG AGA |
| 19 | H4A(+13 +32) | GCA UGA ACU CUU GUG GAU CC |
| 20 | H4D(+04 −16) | CCA GGG UAC UAC UUA CAU UA |
| 21 | H4D(−24 −44) | AUC GUG UGU CAC AGC AUC CAG |
| 22 | H4A(+11 +40) | UGU UCA GGG CAU GAA CUC UUG UGG AUC |
| | | CUU |
| 23 | H3A(+30 +60) | UAG GAG GCG CCU CCC AUC CUG UAG GUC |
| | | ACU G |
| 24 | H3A(+35 +65) | AGG UCU AGG AGG CGC CUC CCA UCC UGU |
| | | AGG U |
| 25 | H3A(+30 +54) | GCG CCU CCC AUC CUG UAG GUC ACU G |
| 26 | H3D(+46 −21) | CUU CGA GGA GGU CUA GGA GGC GCC UC |
| 27 | H3A(+30 +50) | CUC CCA UCC UGU AGG UCA CUG |
| 28 | H3D(+19 −03) | UAC CAG UUU UUG CCC UGU CAG G |
| 29 | H3A(−06 +20) | UCA AUA UGC UGC UUC CCA AAC UGA AA |
| 30 | H3A(+37 +61) | CUA GGA GGC GCC UCC CAU CCU GUA G |
| 31 | H5A(+20 +50) | UUA UGA UUU CCA UCU ACG AUG UCA GUA |
| | | CUU C |
| 32 | H5D(+25 −05) | CUU ACC UGC CAG UGG AGG AUU AUA UUC |
| | | CAA A |
| 33 | H5D(+10 −15) | CAU CAG GAU UCU UAC CUG CCA GUG G |
| 34 | H5A(+10 +34) | CGA UGU CAG UAC UUC CAA UAU UCA C |
| 35 | H5D(−04 −21) | ACC AUU CAU CAG GAU UCU |
| 36 | H5D(+16 −02) | ACC UGC CAG UGG AGG AUU |
| 37 | H5A(−07 +20) | CCA AUA UUC ACU AAA UCA ACC UGU UAA |
| 38 | H5D(+18 −12) | CAG GAU UGU UAC CUG CCA GUG GAG GAU |
| | | UAU |
| 39 | H5A(+05 +35) | ACG AUG UCA GUA CUU CCA AUA UUC ACU |
| | | AAA U |
| 40 | H5A(+15 +45) | AUU UCC AUC UAC GAU GUC AGU ACU UCC |
| | | AAU A |
| 41 | H10A(−05 +16) | CAG GAG CUU CCA AAU GCU GCA |
| 42 | H10A(−05 +24) | CUU GUC UUC AGG AGC UUC CAA AUG CUG CA |
| 43 | H10A(+98 +119) | UCC UCA GCA GAA AGA AGC CAC G |
| 44 | H10A(+130 +149) | UUA GAA AUC UCU CCU UGU GC |
| 45 | H10A(−33 −14) | UAA AUU GGG UGU UAC ACA AU |
| 46 | H11D(+26 +49) | CCC UGA GGC AUU CCC AUC UUG AAU |
| 47 | H11D(+11 −09) | AGG ACU UAC UUG CUU UGU UU |
| 48 | H11A(+118 +140) | CUU GAA UUU AGG AGA UUC AUC UG |
| 49 | H11A(+75 +97) | CAU CUU CUG AUA AUU UUC CUG UU |
| 50 | H12A(+52 +75) | UCU UCU GUU UUU GUU AGC CAG UCA |
| 51 | H12A(−10 +10) | UCU AUG UAA ACU GAA AAU UU |
| 52 | H12A(+11 +30) | UUC UGG AGA UCC AUU AAA AC |
| 53 | H13A(+77 +100) | CAG CAG UUG CGU GAU CUC CAC UAG |
| 54 | H13A(+55 +75) | UUC AUC AAC UAC CAC CAC CAU |
| 55 | H13D(+06 −19) | CUA AGC AAA AUA AUC UGA CCU UAA G |
| 56 | H14A(+37 +64) | CUU GUA AAA GAA CCC AGC GGU CUU CUG U |
| 57 | H14A(+14 +35) | CAU CUA CAG AUG UUU GCC CAU C |
| 58 | H14A(+51 +73) | GAA GGA UGU CUU GUA AAA GAA CC |
| 59 | H14D(−02 +18) | ACC UGU UCU UCA GUA AGA CG |
| 60 | H14D(+14 −10) | CAU GAC ACA CCU GUU CUU CAG UAA |
| 61 | H14A(+61 +80) | CAU UUG AGA AGG AUG UCU UG |
| 62 | H14A(−12 +12) | AUC UCC CAA UAC CUG GAG AAG AGA |
| 63 | H15A(−12 +19) | GCC AUG CAC UAA AAA GGC ACU GCA AGA |
| | | CAU U |
| 64 | H15A(+48 +71) | UCU UUA AAG CCA GUU GUG UGA AUC |
| 65 | H15A(+08 +28) | UUU CUG AAA GCC AUG CAC UAA |
| 66 | H15D(+17 −08) | GUA CAU ACG GCC AGU UUU UGA AGA C |
| 67 | H16A(−12 +19) | CUA GAU CCG CUU UUA AAA CCU GUU AAA |
| | | ACA A |
| 68 | H16A(−06 +25) | UCU UUU CUA GAU CCG CUU UUA AAA CCU |
| | | GUU A |
| 69 | H16A(−06 +19) | CUA GAU CCG CUU UUA AAA CCU GUU A |
| 70 | H16A(+87 +109) | CCG UCU UCU GGG UCA CUG ACU UA |
| 71 | H16A(−07 +19) | CUA GAU CCG CUU UUA AAA CCU GUU AA |
| 72 | H16A(−07 +13) | CCG CUU UUA AAA CCU GUU AA |
| 73 | H16A(+12 +37) | UGG AUU GCU UUU UCU UUU CUA GAU CC |
| 74 | H16A(+92 +116) | CAU GCU UCC GUC UUC UGG GUC ACU G |
| 75 | H16A(+45 +67) | G AUC UUG UUU GAG UGA AUA CAG U |
| 76 | H16A(+105 +126) | GUU AUC CAG CCA UGC UUC CGU C |
| 77 | H16D(+05 −20) | UGA UAA UUG GUA UCA CUA ACC UGU G |
| 78 | H16D(+12 −11) | GUA UCA CUA ACC UGU GCU GUA C |
| 79 | H19A(+35 +53) | CUG CUG GCA UCU UGC AGU U |
| 80 | H19A(+35 +65) | GCC UGA GCU GAU CUG CUG GCA UCU UGC |
| | | AGU U |
| 81 | H20A(+44 +71) | CUG GCA GAA UUC GAU CCA CCG GCU GUU C |
| 82 | H20A(+147 +168) | CAG CAG UAG UUG UCA UCU GCU C |
| 83 | H20A(+185 +203) | UGA UGG GGU GGU GGG UUG G |
| 84 | H20A(−08 +17) | AUC UGC AUU AAC ACC CUC UAG AAA G |
| 85 | H20A(+30 +53) | CCG GCU GUU CAG UUG UUC UGA GGC |
| 86 | H20A(−11 +17) | AUC UGC AUU AAC ACC CUC UAG AAA GAA A |
| 87 | H20D(+08 −20) | GAA GGA GAA GAG AUU CUU ACC UUA CAA A |
| 88 | H20A(+44 +63) | AUU CGA UCC ACC GGC UGU UC |
| 89 | H20A(+149 +168 | CAG CAG UAG UUG UCA UCU GC |
| 90 | H21A(−06 +16) | GCC GGU UGA CUU CAU CCU GUG C |
| 91 | H21A(+85 +106) | CUG CAU CCA GGA ACA UGG GUC C |
| 92 | H21A(+85 +108 | GUC UGC AUC CAG GAA CAU GGG UC |
| 93 | H21A(+08 +31) | GUU GAA GAU CUG AUA GCC GGU UGA |
| 94 | H21D(+18 −07) | UAC UUA CUG UCU GUA GCU CUU UCU |
| 95 | H22A(+22 +45) | CAC UCA UGG UCU CCU GAU AGC GCA |
| 96 | H22A(+125 +106) | CUG CAA UUC CCC GAG UCU CUG C |
| 97 | H22A(+47 +69) | ACU GCU GGA CCC AUG UCC UGA UG |
| 98 | H22A(+80 +101) | CUA AGU UGA GGU AUG GAG AGU |
| 99 | H22D(+13 −11) | UAU UCA CAG ACC UGC AAU UCC CC |
| 100 | H23A(+34 +59) | ACA GUG GUG CUG AGA UAG UAU AGG CC |
| 101 | H23A(+18 +39) | UAG GCC ACU UUG UUG CUC UUG C |
| 102 | H23A(+72 +90) | UUC AGA GGG CGC UUU CUU C |
| 103 | H24A(+48 +70) | GGG CAG GCC AUU CCU CCU UCA GA |
| 104 | H24A(−02 +22) | UCU UCA GGG UUU GUA UGU GAU UCU |
| 105 | H25A(+9 +36) | CUG GGC UGA AUU GUC UGA AUA UCA CUG |
| 106 | H25A(+131 +156) | CUG UUG GCA CAU GUG AUC CCA CUG AG |
| 107 | H25D(+16 −08) | GUC UAU ACC UGU UGG CAC AUG UGA |
| 108 | H26A(+132 +156) | UGC UUU CUG UAA UUC AUC UGG AGU U |
| 109 | H26A(−07 +19) | CCU CCU UUC UGG CAU AGA CCU UCC AC |
| 110 | H26A(+68 +92) | UGU GUC AUC CAU UCG UGC AUC UCU G |
| 111 | H27A(+82 +106) | UUA AGG CCU CUU GUG CUA CAG GUG G |
| 112 | H27A(−4 +19) | GGG GCU CUU CUU UAG CUC UCU GA |
| 113 | H27D(+19 −03) | GAC UUC CAA AGU CUU GCA UUU C |
| 114 | H28A(−05 +19) | GCC AAC AUG CCC AAA CUU CCU AAG |
| 115 | H28A(+99 +124) | CAG AGA UUU CCU CAG CUC CGC CAG GA |
| 116 | H28D(+16 −05) | CUU ACA UCU AGC ACC UCA GAG |
| 117 | H29A(+57 +81) | UCC GCC AUC UGU UAG GGU CUG UGC C |
| 118 | H29A(+18 +42) | AUU UGG GUU AUC CUC UGA AUG UCG C |
| 119 | H29D(+17 −05) | CAU ACC UCU UCA UGU AGU UCC C |
| 120 | H30A(+122 +147) | CAU UUG AGC UGC GUC CAC CUU GUC UG |
| 121 | H30A(+25 +50) | UCC UGG GCA GAC UGG AUG CUC UGU UC |
| 122 | H30D(+19 −04) | UUG CCU GGG CUU CCU GAG GCA UU |
| 123 | H31D(+06 −18) | UUC UGA AAU AAC AUA UAC CUG UGC |
| 124 | H31D(+03 −22) | UAG UUU CUG AAA UAA CAU AUA CCU G |
| 125 | H31A(+05 +25) | GAC UUG UCA AAU CAG AUU GGA |
| 126 | H31D(+04 −20) | GUU UCU GAA AUA ACA UAU ACC UGU |
| 127 | H32D(+04 −16) | CAC CAG AAA UAC AUA CCA CA |
| 128 | H32A(+151 +170) | CAA UGA UUU AGC UGU GAC UG |
| 129 | H32A(+10 +32) | CGA AAC UUC AUG GAG ACA UCU UG |
| 130 | H32A(+49 +73) | CUU GUA GAC GCU GCU CAA AAU UGG C |
| 131 | H33D(+09 −11) | CAU GCA CAC ACC UUU GCU CC |
| 132 | H33A(+53 +76) | UCU GUA CAA UCU GAC GUC CAG UCU |
| 133 | H33A(+30 +56) | GUC UUU AUC ACC AUU UCC ACU UCA GAC |
| 134 | H33A(+64 +88) | CCG UCU GCU UUU UCU GUA CAA UCU G |
| 135 | H34A(+83 +104) | UCC AUA UCU GUA GCU GCC AGC C |
| 136 | H34A(+143 +165) | CCA GGC AAC UUC AGA AUC CAA AU |
| 137 | H34A(−20 +10) | UUU CUG UUA CCU GAA AAG AAU UAU AAU |
| | | GAA |
| 138 | H34A(+46 +70) | CAU UCA UUU CCU UUC GCA UCU UAC G |
| 139 | H34A(+95 +120) | UGA UCU CUU UGU CAA UUC CAU AUC UG |
| 140 | H34D(+10 −20) | UUC AGU GAU AUA GGU UUU ACC UUU CCC |
| | | CAG |
| 141 | H34A(+72 +96) | CUG UAG CUG CCA GCC AUU CUG UCA AG |
| 142 | H35A(+141 +161) | UCU UCU GCU CGG GAG GUG ACA |
| 143 | H35A(+116 +135) | CCA GUU ACU AUU CAG AAG AC |
| 144 | H35A(+24 +43) | UCU UCA GGU GCA CCU UCU GU |
| 145 | H36A(+26 +50) | UGU GAU GUG GUC CAC AUU CUG GUC A |
| 146 | H36A(−02 +18) | CCA UGU GUU UCU GGU AUU CC |
| 147 | H37A(+26 +50) | CGU GUA GAG UCC ACC UUU GGG CGU A |
| 148 | H37A(+82 +105) | UAC UAA UUU CCU GCA GUG GUC ACC |
| 149 | H37A(+134 +157) | UUC UGU GUG AAA UGG CUG CAA AUC |
| 150 | H38A(−01 +19) | CCU UCA AAG GAA UGG AGG CC |
| 151 | H38A(+59 +83) | UGC UGA AUU UCA GCC UCC AGU GGU U |
| 152 | H38A(+88 +112) | UGA AGU CUU CCU CUU UCA GAU UCA C |
| 153 | H39A(+62 +85) | CUG GCU UUC UCU CAU CUG UGA UUC |
| 154 | H39A(+39 +58) | GUU GUA AGU UGU CUC CUC UU |
| 155 | H39A(+102 +121) | UUG UCU GUA ACA GCU GCU GU |
| 156 | H39D(+10 −10) | GCU CUA AUA CCU UGA GAG CA |
| 157 | H40A(−05 +17) | CUU UGA GAC CUC AAA UCC UGU U |
| 158 | H40A(+129 +153) | CUU UAU UUU CCU UUC AUC UCU GGG C |
| 159 | H42A(−04 +23) | AUC GUU UCU UCA CGG ACA GUG UGC UGG |
| 160 | H42A(+86 +109) | GGG CUU GUG AGA CAU GAG UGA UUU |
| 161 | H42D(+19 −02) | A CCU UCA GAG GAC UCC UCU UGC |
| 162 | H43D(+10 −15) | UAU GUG UUA CCU ACC CUU GUC GGU C |
| 163 | H43A(+101 +120) | GGA GAG AGC UUC CUG UAG CU |
| 164 | H43A(+78 +100) | UCA CCC UUU CCA CAG GCG UUG CA |
| 165 | H44A(+85 +104) | UUU GUG UCU UUC UGA GAA AC |
| 166 | H44D(+10 −10) | AAA GAC UUA CCU UAA GAU AC |
| 167 | H44A(−06 +14) | AUC UGU CAA AUC GCC UGC AG |
| 168 | H46D(+16 −04) | UUA CCU UGA CUU GCU CAA GC |
| 169 | H46A(+90 +109) | UCC AGG UUC AAG UGG GAU AC |
| 170 | H47A(+76 +100) | GCU CUU CUG GGC UUA UGG GAG CAC U |
| 171 | H47D(+25 −02) | ACC UUU AUC CAC UGG AGA UUU GUC UGC |
| 172 | H47A(−9+ 12) | UUC CAC CAG UAA CUG AAA CAG |
| 173 | H50A(+02 +30) | CCA CUC AGA GCU CAG AUC UUC UAA CUU CC |
| 174 | H50A(+07 +33) | CUU CCA CUC AGA GCU CAG AUC UUC UAA |
| 175 | H50D(+07 −18) | GGG AUC CAG UAU ACU UAC AGG CUC C |
| 176 | H51A(−01 +25) | ACC AGA GUA ACA GUC UGA GUA GGA GC |
| 177 | H51D(+16 −07) | CUC AUA CCU UCU GCU UGA UGA UC |
| 178 | H51A(+111 +134) | UUC UGU CCA AGC CCG GUU GAA AUC |
| 179 | H51A(+61 +90) | ACA UCA AGG AAG AUG GCA UUU CUA GUU |
| | | UGG |
| 180 | H51A(+66 +90) | ACA UCA AGG AAG AUG GCA UUU CUA G |
| 181 | H51A(+66 +95) | CUC CAA CAU CAA GGA AGA UGG CAU UUC |
| | | UAG |
| 182 | H51D(+08 −17) | AUC AUU UUU UCU CAU ACC UUC UGC U |
| 183 | H51A/D(+08 −17) | AUC AUU UUU UCU CAU ACC UUC UGC UAG |
| | & (−15+) | GAG CUA AAA |
| 184 | H51A(+175 +195) | CAC CCA CCA UCA CCC UCU GUG |
| 185 | H51A(+199 +220) | AUC AUC UCG UUG AUA UCC UCA A |
| 186 | H52A(−07 +14) | UCC UGC AUU GUU GCC UGU AAG |
| 187 | H52A(+12 +41) | UCC AAC UGG GGA CGC CUC UGU UCC AAA |
| | | UCC |
| 188 | H52A(+17 +37) | ACU GGG GAC GCC UCU GUU CCA |
| 189 | H52A(+93 +112) | CCG UAA UGA UUG UUC UAG CC |
| 190 | H52D(+05 −15) | UGU UAA AAA ACU UAC UUC GA |
| 191 | H53A(+45 +69) | CAU UCA ACU GUU GCC UCC GGU UCU G |
| 192 | H53A(+39 +62) | CUG UUG CCU CCG GUU CUG AAG GUG |
| 193 | H53A(+39 +69) | CAU UCA ACU GUU GCC UCC GGU UCU GAA |
| | | GGU G |
| 194 | H53D(+14 −07) | UAC UAA CCU UGG UUU CUG UGA |
| 195 | H53A(+23 +47) | CUG AAG GUG UUC UUG UAC UUC AUC C |
| 196 | H53A(+150 +176) | UGU AUA GGG ACC CUC CUU CCA UGA CUC |
| 197 | H53D(+20 −05) | CUA ACC UUG GUU UCU GUG AUU UUC U |
| 198 | H53D(+09 −18) | GGU AUC UUU GAU ACU AAC CUU GGU UUC |
| 199 | H53A(−12 +10) | AUU CUU UCA ACU AGA AUA AAA G |
| 200 | H53A(−07 +18) | GAU UCU GAA UUC UUU CAA CUA GAA U |
| 201 | H53A(+07 +26) | AUC CCA CUG AUU CUG AAU UC |
| 202 | H53A(+124 +145) | UUG GCU CUG GCC UGU CCU AAG A |
| 203 | H46A(+86 +115) | CUC UUU UCC AGG UUC AAG UGG GAU ACU |
| | | AGC |
| 204 | H46A(+107 +137) | CAA GCU UUU CUU UUA GUU GCU GCU CUU |
| | | UUC C |
| 205 | H46A(−10 +20) | UAU UCU UUU GUU CUU CUA GCC UGG AGA |
| | | AAG |
| 206 | H46A(+50 +77) | CUG CUU CCU CCA ACC AUA AAA CAA AUU C |
| 207 | H45A(−06 +20) | CCA AUG CCA UCC UGG AGU UCC UGU AA |
| 208 | H45A(+91 +110) | UCC UGU AGA AUA CUG GCA UC |
| 209 | H45A(+125 +151) | UGC AGA CCU CCU GCC ACC GCA GAU UCA |
| 210 | H45D(+16 −04) | CUA CCU CUU UUU UCU GUC UG |
| 211 | H45A(+71 +90) | UGU UUU UGA GGA UUG CUG AA |

TABLE 1B
Description of a cocktail of 2′-O-methyl phosphorothioate antisense
oligonucleotides that have been used to date to study induced exon
skipping during the processing of the dystrophin pre-mRNA.

| SEQ | | |
|---|---|---|
| ID | SEQUENCE | NUCLEOTIDE SEQUENCE (5′-3′) |
| 81 | H20A(+44 +71) | CUG GCA GAA UUC GAU CCA CCG GCU |
| 82 | H20A(+147 +168) | GUU C |
| | | CAG CAG UAG UUG UCA UCU GCU C |
| 80 | H19A(+35 +65) | GCC UGA GCU GAU CUG CUG GCA UCU |
| 81 | H20A(+44 +71) | UGC |
| 82 | H20A(+147 +168) | AGU U |
| | | CUG GCA GAA UUC GAU CCA CCG GCU |
| | | GUU C |
| | | CAG CAG UAG UUG UCA UCU GCU C |
| 194 | H53D(+14 −07) | UAC UAA CCU UGG UUU CUG UGA |
| 195 | H53A(+23 +47) | CUG AAG GUG UUC UUG UAC UUC AUC C |
| 196 | H53A(+150 +175) | UGU AUA GGG ACC CUC CUU CCA UGA |
| | | CUC |

TABLE 1C
Description of a “weasel” of 2′-O-methyl phosphorothioate antisense
oligonucleotides that have been used to date to study induced exon
skipping during the processing of the dystrophin pre-mRNA.

| SEQ | | |
|---|---|---|
| ID | SEQUENCE | NUCLEOTIDE SEQUENCE (5′-3′) |
| 81 | H20A(+44 +71)- | CUG GCA GAA UUC GAU CCA CCG GCU GUU C- |
| 82 | H20A(+147 +168) | CAG CAG UAG UUG UCA UCU GCU C |
| 80 | H19A(+35 +65)- | GCC UGA GCU GAU CUG CUG GCA UCU UGC |
| | | AGU U |
| 88 | H20A(+44 +63)- | -AUU CGA UCC ACC GGC UGU UC- |
| 79 | H20A(+149 +168) | CUG CUG GCA UCU UGC AGU U |
| 80 | H19A(+35 +65)- | GCC UGA GCU GAU CUG CUG GCA UCU UGC |
| | | AGU U |
| 88 | H20A(+44 +63) | -AUU CGA UCC ACC GGC UGU UC- |
| 80 | H19A(+35 +65)- | GCC UGA GCU GAU CUG CUG GCA UCU UGC |
| | | AGU U |
| 79 | H20A(+149 +168) | -CUG CUG GCA UCU UGC AGU U |
| 138 | H34A(+46 +70)- | CAU UCA UUU CCU UUC GCA UCU UAC G- |
| 139 | H34A(+94 +120) | UGA UCU CUU UGU CAA UUC CAU AUC UG |
| 124 | H31D(+03 −22)- | UAG UUU CUG AAA UAA CAU AUA CCU G- |
| | UU- | UU- |
| 144 | H35A(+24 +43) | UCU UCA GGU GCA CCU UCU GU |
| 195 | H53A(+23 +47)- | CUG AAG GUG UUC UUG UAC UUC AUC C- |
| | AA- | |
| 196 | H53A(+150 +175)- | UGU AUA GGG ACC CUC CUU CCA UGA CUC- |
| | AA- | AA- |
| 194 | H53D(+14 −07) | UAC UAA CCU UGG UUU CUG UGA |
| — | Aimed at exons | CAG CAG UAG UUG UCA UCU GCU CAA CUG |
| 212 | 19/20/20 | GCA GAA UUC GAU CCA CCG GCU GUU CAA |
| | | GCC UGA GCU GAU CUG CUC GCA UCU |
| | | UGC AGU |

### DETAILED DESCRIPTION OF THE INVENTION

### General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

Sequence identity numbers (SEQ ID NO:) containing nucleotide and amino acid sequence information included in this specification are collected at the end of the description and have been prepared using the programme PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense molecules nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:

H#A/D(x:y).

The first letter designates the species (e.g. H: human, M: murine, C: canine) “#” designates target dystrophin exon number.

“A/D” indicates acceptor or donor splice site at the beginning and end of the exon, respectively.

(x y) represents the annealing coordinates where “−” or “+” indicate intronic or exonic sequences respectively, As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used necessarily herein the term “derived” and “derived from” shall be taken to indicate that a specific integer may be obtained from a particular source albeit not directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DESCRIPTION OF THE PREFERRED EMBODIMENT

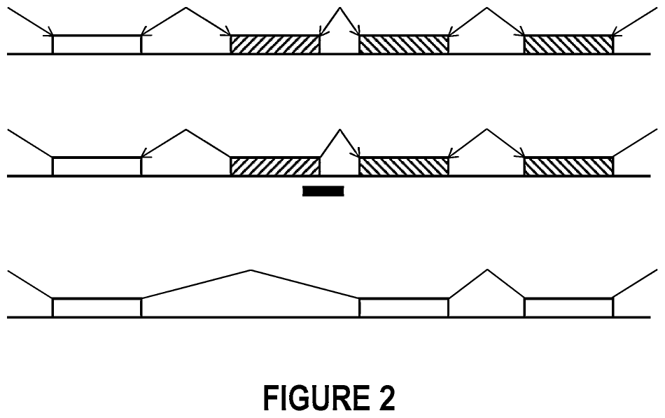

When antisense molecule(s) are targeted to nucleotide sequences involved in splicing in exons within pre-mRNA sequences, normal splicing of the exon may be inhibited causing the splicing machinery to by-pass the entire mutated exon from the mature mRNA. The concept of antisense oligonucleotide induced exon skipping is shown in FIG. 2. In many genes, deletion of an entire exon would lead to the production of a non-functional protein through the loss of important functional domains or the disruption of the reading frame. In some proteins, however, it is possible to shorten the protein by deleting one or more exons, without disrupting the reading frame, from within the protein without seriously altering the biological activity of the protein. Typically, such proteins have a structural role and or possess functional domains at their ends. The present invention describes antisense molecules capable of binding to specified dystrophin pre-mRNA targets and re-directing processing of that gene.

### Antisense Molecules

According to a first aspect of the invention, there is provided antisense molecules capable of binding to a selected target to induce exon skipping. To induce exon skipping in exons of the Dystrophin gene transcript, the antisense molecules are preferably selected from the group of compounds shown in Table 1A. There is also provided a combination or “cocktail” of two or more antisense oligonucleotides capable of binding to a selected target to induce exon skipping. To induce exon skipping in exons of the Dystrophin gene transcript, the antisense molecules in a “cocktail” are preferably selected from the group of compounds shown in Table 1B. Alternatively, exon skipping may be induced by antisense oligonucleotides joined together “weasels” preferably selected from the group of compounds shown in Table 1C.

Designing antisense molecules to completely mask consensus splice sites may not necessarily generate any skipping of the targeted exon. Furthermore, the inventors have discovered that size or length of the antisense oligonucleotide itself is not always a primary factor when designing antisense molecules. With some targets such as exon 19, antisense oligonucleotides as short as 12 bases were able to induce exon skipping, albeit not as efficiently as longer (20-31 bases) oligonucleotides. In some other targets, such as murine dystrophin exon 23, antisense oligonucleotides only 17 residues long were able to induce more efficient skipping than another overlapping compound of 25 nucleotides.

The inventors have also discovered that there does not appear to be any standard motif that can be blocked or masked by antisense molecules to redirect splicing. In some exons, such as mouse dystrophin exon 23, the donor splice site was the most amenable to target to re-direct skipping of that exon. It should be noted that designing and testing a series of exon 23 specific antisense molecules to anneal to overlapping regions of the donor splice site showed considerable variation in the efficacy of induced exon skipping. As reported in Mann et al., (2002) there was a significant variation in the efficiency of bypassing the nonsense mutation depending upon antisense oligonucleotide annealing (“Improved antisense oligonucleotide induced exon skipping in the mdx mouse model of muscular dystrophy”. J Gen Med 4:644-654). Targeting the acceptor site of exon 23 or several internal domains was not found to induce any consistent exon 23 skipping.

In other exons targeted for removal, masking the donor splice site did not induce any exon skipping. However, by directing antisense molecules to the acceptor splice site (human exon 8 as discussed below), strong and sustained exon skipping was induced. It should be noted that removal of human exon 8 was tightly linked with the co-removal of exon 9. There is no strong sequence homology between the exon 8 antisense oligonucleotides and corresponding regions of exon 9 so it does not appear to be a matter of cross reaction. Rather the splicing of these two exons is inextricably linked. This is not an isolated instance as the same effect is observed in canine cells where targeting exon 8 for removal also resulted in the skipping of exon 9. Targeting exon 23 for removal in the mouse dystrophin pre-mRNA also results in the frequent removal of exon 22 as well. This effect occurs in a dose dependent manner and also indicates close coordinated processing of 2 adjacent exons.

In other targeted exons, antisense molecules directed at the donor or acceptor splice sites did not induce exon skipping while annealing antisense molecules to intra-exonic regions (i.e. exon splicing enhancers within human dystrophin exon 6) was most efficient at inducing exon skipping. Some exons, both mouse and human exon 19 for example, are readily skipped by targeting antisense molecules to a variety of motifs. That is, targeted exon skipping is induced after using antisense oligonucleotides to mask donor and acceptor splice sites or exon splicing enhancers.

To identify and select antisense oligonucleotides suitable for use in the modulation of exon skipping, a nucleic acid sequence whose function is to be modulated must first be identified. This may be, for example, a gene (or mRNA transcribed form the gene) whose expression is associated with a particular disorder or disease state, or a nucleic acid molecule from an infectious agent. Within the context of the present invention, preferred target site(s) are those involved in mRNA splicing (i.e. splice donor sites, splice acceptor sites, or exonic splicing enhancer elements). Splicing branch points and exon recognition sequences or splice enhancers are also potential target sites for modulation of mRNA splicing.

Preferably, the present invention aims to provide antisense molecules capable of binding to a selected target in the dystrophin pre-mRNA to induce efficient and consistent exon skipping. Duchenne muscular dystrophy arises from mutations that preclude the synthesis of a functional dystrophin gene product. These Duchenne muscular dystrophy gene defects are typically nonsense mutations or genomic rearrangements such as deletions, duplications or micro-deletions or insertions that disrupt the reading frame. As the human dystropbin gene is a large and complex gene with the 79 exons being spliced together to generate a mature mRNA with an open reading frame of approximately 11,000 bases, there are many positions where these mutations can occur. Consequently, a comprehensive antisense oligonucleotide based therapy to address many of the different disease-causing mutations in the dystrophin gene will require that many exons can be targeted for removal during the splicing process.

Within the context of the present invention, preferred target site(s) are those involved in mRNA splicing (i.e. splice donor sites, splice acceptor sites or exonic splicing enhancer elements). Splicing branch points and exon recognition sequences or splice enhancers are also potential target sites for modulation of mRNA splicing.

The oligonucleotide and the DNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the oligonucleotide and the DNA or RNA target. It is understood in the art that the sequence of an antisense molecule need not be 100% complementary to that of its target sequence to be specifically hybridisable. An antisense molecule is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

While the above method may be used to select antisense molecules capable of deleting any exon from within a protein that is capable of being shortened without affecting its biological function, the exon deletion should not lead to a reading frame shift in the shortened transcribed mRNA. Thus, if in a linear sequence of three exons the end of the first exon encodes two of three nucleotides in a codon and the next exon is deleted then the third exon in the linear sequence must start with a single nucleotide that is capable of completing the nucleotide triplet for a codon. If the third exon does not commence with a single nucleotide there will be a reading frame shift that would lead to the generation of truncated or a non-functional protein.

It wilt be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligonucleotides may need to be selected by the method of the invention wherein each is directed to a different region responsible for inducing splicing in the exons that are to be deleted.

The length of an antisense molecule may vary so long as it is capable of binding selectively to the intended location within the pre-mRNA molecule. The length of such sequences can be determined in accordance with selection procedures described herein. Generally, the antisense molecule will be from about 10 nucleotides in length up to about 50 nucleotides in length. It will be appreciated however that any length of nucleotides within this range may be used in the method. Preferably, the length of the antisense molecule is between 17 to 30 nucleotides in length.

In order to determine which exons can be connected in a dystrophin gene, reference should be made to an exon boundary map. Connection of one exon with another is based on the exons possessing the same number at the 3′ border as is present at the 5′ border of the exon to which it is being connected. Therefore, if exon 7 were deleted, exon 6 must connect to either exons 12 or 18 to maintain the reading frame. Thus, antisense oligonucleotides would need to be selected which redirected splicing for exons 7 to 11 in the first instance or exons 7 to 17 in the second instance. Another and somewhat simpler approach to restore the reading frame around an exon 7 deletion would be to remove the two flanking exons. Induction of exons 6 and 8 skipping should result in an in-frame transcript with the splicing of exons 5 to 9. In practise however, targeting exon 8 for removal from the pre-mRNA results in the co-removal of exon 9 so the resultant transcript would have exon 5 joined to exon 10. The inclusion or exclusion of exon 9 does not alter the reading frame. Once the antisense molecules to be tested have been identified, they are prepared according to standard techniques known in the art. The most common method for producing antisense molecules is the methylation of the 2′ hydroxyribose position and the incorporation of a phosphorothioate backbone produces molecules that superficially resemble RNA but that are much more resistant to nuclease degradation.

To avoid degradation of pre-mRNA during duplex formation with the antisense molecules, the antisense molecules used in the method may be adapted to minimise or prevent cleavage by endogenous RNase H. This property is highly preferred as the treatment of the RNA with the unmethylated oligonucleotides either intracellularly or in crude extracts that contain RNase H leads to degradation of the pre-mRNA: antisense oligonucleotide duplexes. Any form of modified antisense molecules that is capable of by-passing or not inducing such degradation may be used in the present method. An example of antisense molecules which when duplexed with RNA are not cleaved by cellular R.Nase His 2′-O-methyl derivatives. 2′-O-methyl-oligoribonucleotides are very stable in a cellular environment and in animal tissues, and their duplexes with RNA have higher Tm values than their ribo-or deoxyribo-counterparts.

Antisense molecules that do not activate RNase H can be made in accordance with known techniques (see, e.g., U.S. Pat. No. 5,149,797). Such antisense molecules, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense molecules that do not activate RNase H are available. For example, such antisense molecules may be oligonucleotides wherein at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

While antisense oligonucleotides are a preferred form of the antisense molecules, the present invention comprehends other oligomeric antisense molecules, including but not limited to oligonucleotide mimetics such as are described below.

Specific examples of preferred antisense compounds useful in this invention include oligonucleotides containing modified backbones or non-natural inter-nucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their inter-nucleoside backbone can also be considered to be oligonucleosides.

In other preferred oligonucleotide mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Modified oligonucleotides may also contain one or more substituted sugar moieties. Oligonucleotides may also include nucleobase (often referred to in the art simply as “base”) modifications or substitutions. Certain nucleo-bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary far all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

### Methods of Manufacturing Antisense Molecules

The antisense molecules used in accordance with this invention may be conveniently and routinely made through the well-known technique of solid phase synthesis. Equipment for such synthesis is sold by several vendors including, for example, Applied Biosystems (Foster City, Calif.). One method for synthesising oligonucleotides on a modified solid support is described in U.S. Pat. No. 4,458,066.

Any other means for such synthesis known in the art may additionally or alternatively be employed. It is well known to use similar techniques to prepare oligonucleotides such as the phosphorothioates˜and alkylated derivatives. In one such automated embodiment, diethyl-phosphoramidites are used as starting materials and may be synthesized as described by Beaucage, et al., (1981) Tetrahedron Letters, 22:1859-1862.

The antisense molecules of the invention are synthesised in vitro and do not include antisense compositions of biological origin, or genetic vector constructs designed to direct the in vivo synthesis of antisense molecules. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

### Therapeutic Agents

The present invention also can be used as a prophylactic or therapeutic, which may be utilised for the purpose of treatment of a genetic disease.

Accordingly, in one embodiment the present invention provides antisense molecules that bind to a selected target in the dystrophin pre-mRNA to induce efficient and consistent exon skipping described herein in a therapeutically effective amount admixed with a pharmaceutically acceptable carrier, diluent, or excipient.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

In a more specific form of the invention there are provided pharmaceutical compositions comprising therapeutically effective amounts of an antisense molecule together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Martin, Remington's Pharmaceutical Sciences, 18th Ed. (1990, Mack Publishing Co., Easton, PA 18042) pages 1435-1712 that are herein incorporated by reference. The compositions may be prepared in liquid form, or may be in dried powder, such as lyophilised form.

It will be appreciated that pharmaceutical compositions provided according to the present invention may be administered by any means known in the art. Preferably, the pharmaceutical compositions for administration are administered by injection, orally, or by the pulmonary, or nasal route. The antisense molecules are more preferably delivered by intravenous, intra-arterial, intraperitoneal, intramuscular, or subcutaneous routes of administration.

### Antisense Molecule Based Therapy

Also addressed by the present invention is the use of antisense molecules of the present invention, for manufacture of a medicament for modulation of a genetic disease.

The delivery of a therapeutically useful amount of antisense molecules may be achieved by methods previously published. For example, intracellular delivery of the antisense molecule may be via a composition comprising an admixture of the antisense molecule and an effective amount of a block copolymer. An example of this method is described in US patent application US 20040248833.

Other methods of delivery of antisense molecules to the nucleus are described in Mann C J et al., (2001) [“Antisense-induced exon skipping and the synthesis of dystrophin in the mdx mouse”. Proc., Natl. Acad. Science, 98(1) 42-47J and in Gebski etal., (2003). Human Molecular Genetics, 12(15): 1801-1811.

A method for introducing a nucleic acid molecule into a cell by way of an expression vector either as naked DNA or complexed to lipid carriers, is described in US patent U.S. Pat. No. 6,806,084.

It may be desirable to deliver the antisense molecule in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations.

Liposomes are artificial membrane vesicles which are useful as delivery vehicles in vitro and in vivo. These formulations may have net cationic, anionic or neutral charge characteristics and are useful characteristics with in vitro, in vivo and ex vivo delivery methods. It has been shown that large unilamellar vesicles (LUV), which range in size from 0.2-4.0 .PHI.m can encapsulate a substantial percentage of an aqueous buffer containing large macromolecules. RNA, and DNA can be encapsulated within the aqueous interior and be delivered to cells in a biologically active form (Fraley, et al., Trends Biochem. Sci., 6:77, 1981).

In order for a liposome to be an efficient gene transfer vehicle, the following characteristics should be present: (1) encapsulation of the antisense molecule of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Alternatively, the antisense construct may be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral or transdermal administration.

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280).

These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

The antisense molecules of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, bydrobromic acid, sulfuric acid, phosphoric acid, nitric acid and the like; (c) salts formed with organic acids such as, for example, acetic acid, oxalic acid, tartaric acid, succinic acid, malefic acid, fumaric acid, gluconic acid, citric acid, malic acid, ascorbic acid, benzoic acid, tannic acid, palmitic acid, alginic acid, polygiutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and to mucous membranes including rectal delivery), pulmonary, e.g., by inhalation or insufflation of powders or aerosols, (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration, Oligonucleotides with at least one 2′-O-methoxyethyl modification are believed to be particularly useful for oral administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

### Kits of the Invention

The invention also provides kits for treatment of a patient with a genetic disease which kit comprises at least an antisense molecule, packaged in a suitable container, together with instructions for its use.

In a preferred embodiment, the kits will contain at least one antisense molecule as shown in Table 1A, or a cocktail of antisense molecules as shown in Table 1B or a “weasel” compound as shown in Table 1C. The kits may also contain peripheral reagents such as buffers, stabilizers, etc.

Those of ordinary skill in the field should appreciate that applications of the above method has wide application for identifying antisense molecules suitable for use in the treatment of many other diseases.

### EXAMPLES

The following Examples serve to more fully describe the manner of using the above-described invention, as well as to set forth the best modes contemplated for carrying out various aspects of the invention. It is understood that these Examples in no way serve to limit the true scope of this invention, but rather are presented for illustrative purposes. The references cited herein are expressly incorporated by reference.

Methods of molecular cloning, immunology and protein chemistry, which are not explicitly described in the following examples, are reported in the literature and are known by those skilled in the art. General texts that described conventional molecular biology, microbiology, and recombinant DNA techniques within the skill of the art, included, for example: Sambrook et al, Molecular Cloning: A Laboratory Manual, Second Edition, Cold Spring Harbor Laboratory Press, Cold Spring Harbor, New York (1989); Glover ed., DNA Cloning: A Practical Approach, Volumes I and II, MRL Press, Ltd., Oxford, U.K. (1985); and Ausubel, F., Brent, R., Kingston, R. E., Moore, D. D., Seidman, J. G., Smith, J. A., Struhl, K. Current Protocols in Molecular Biology. Greene Publishing Associates/Wiley Intersciences, New York (2002).

### Determining Induced Exon Skipping in Human Muscle Cells

Attempts by the inventors to develop a rational approach in antisense molecules design were not completely successful as there did not appear to be a consistent trend that could be applied to all exons. As such, the identification of the most effective and therefore most therapeutic antisense molecules compounds has been the result of empirical studies.

These empirical studies involved the use of computer programs to identify motifs potentially involved in the splicing process. Other computer programs were also used to identify regions of the pre-mRNA which may not have had extensive secondary structure and therefore potential sites for annealing of antisense molecules. Neither of these approaches proved completely reliable in designing antisense oligonucleotides for reliable and efficient induction of exon skipping.

Annealing sites on the human dystrophin pre-mRNA were selected for examination, initially based upon known or predicted motifs or regions involved in splicing. 2OMe antisense oligonucleotides were designed to be complementary to the target sequences under investigation and were synthesised on an Expedite 8909 Nucleic Acid Synthesiser. Upon completion of synthesis, the oligonucleotides were cleaved from the support column and de-protected in ammonium hydroxide before being desalted. The quality of the oligonucleotide synthesis was monitored by the intensity of the trityl signals upon each deprotection step during the synthesis as detected in the synthesis log. The concentration of the antisense oligonucleotide was estimated by measuring the absorbance of a diluted aliquot at 260 nm.

Specified amounts of the antisense molecules were then tested for their ability to induce exon skipping in an in vitro assay, as described below.

Briefly, normal primary myoblast cultures were prepared from human muscle biopsies obtained after informed consent. The cells were propagated and allowed to differentiate into myotubes using standard culturing techniques. The cells were then transfected with the antisense oligonucleotides by delivery of the oligonucleotides to the dells as cationic lipoplexes, mixtures of antisense molecules or cationic liposome preparations.

The cells were then allowed to grow for another 24 hours, after which total RNA was extracted and molecular analysis commenced. Reverse transcriptase amplification (RT-PCR) was undertaken to study the targeted regions of the dystrophin pre-mRNA or induced exonic re-arrangements.

For example, in the testing of an antisense molecule for inducing exon 19 skipping the RT-PCR test scanned several exons to detect involvement of any adjacent exons. For example, when inducing skipping of exon 19, RT-PCR was carried out with primers that amplified across exons 17 and 21. Amplifications of even larger products in this area (i.e. exons 13-26) were also carried out to ensure that there was minimal amplification bias for the shorter induced skipped transcript. Shorter or exon skipped products tend to be amplified more efficiently and may bias the estimated of the normal and induced transcript.

The sizes of the amplification reaction products were estimated on an agarose gel and compared against appropriate size standards. The final confirmation of identity of these products was carried out by direct DNA sequencing to establish that the correct or expected exon junctions have been maintained.

Once efficient exon skipping had been induced with one antisense molecule, subsequent overlapping antisense molecules may be synthesized and then evaluated in the assay as described above. Our definition of an efficient antisense molecule is one that induces strong and sustained exon skipping at transfection concentrations in the order of 300 nM or less.

### Antisense Oligonucleotides Directed at Exon 8

Antisense oligonucleotides directed at exon 8 were prepared and tested for their ability to induce exon skipping in human muscle cells using similar methods as described above.

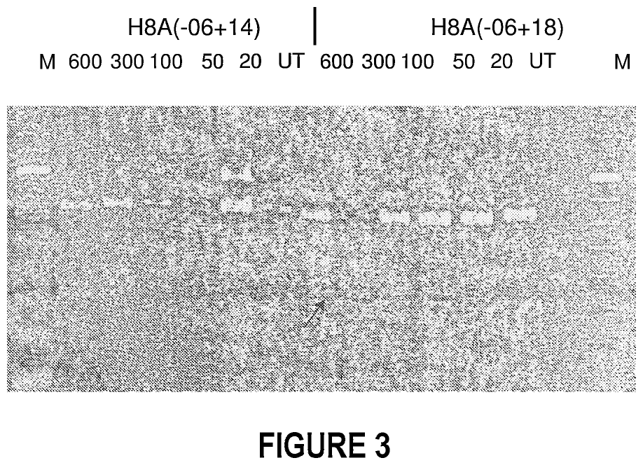

FIG. 3 shows differing efficiencies of two antisense molecules directed at exon 8 acceptor splice site. H8A(−06+18) [SEQ ID NO:1], which anneals to the last 6 bases of intron 7 and the first 18 bases of exon 8, induces substantial exon 8 and 9 skipping when delivered into cells at a concentration of 20 nM. The shorter antisense molecule, H8A(−06+14) [SEQ ID NO: 4] was only able to induce exon 8 and 9 skipping at 300 nM, a concentration some 15 fold higher than H8A(−06+18), which is the preferred antisense molecule,

This data shows that some particular antisense molecules induce efficient exon skipping while another antisense molecule, which targets a near-by or overlapping region, can be much less efficient. Titration studies show one compound is able to induce targeted exon skipping at 20 nM while the less efficient antisense molecules only induced exon skipping at concentrations of 300 nM and above. Therefore, we have shown that targeting of the antisense molecules to motifs involved in the splicing process plays a crucial role in the overall efficacy of that compound.

Efficacy refers to the ability to induce consistent skipping of a target exon. However, sometimes skipping of the target exons is consistently associated with a flanking exon. That is, we have found that the splicing of some exons is tightly linked. For example, in targeting exon 23 in the mouse model of muscular dystrophy with antisense molecules directed at the donor site of that exon, dystrophin transcripts missing exons 22 and 23 are frequently detected. As another example, when using an antisense molecule directed to exon 8 of the human dystrophin gene, all induced transcripts are missing both exons 8 and 9. Dystrophin transcripts missing only exon 8 are not observed.

Table 2 below discloses antisense molecule sequences that induce exon 8 (and 9) skipping.

TABLE 2

| | Antisense | | |
|---|---|---|---|
| | Oligonucleotide | | Ability to induce |
| SEQ ID | name | Sequence | skipping |
| 1 | H8A(−06 +18) | 5′-GAU AGG UGG UAU CAA | Very strong to 20 |
| | | CAU CUG UAA | nM |
| 2 | H8A (−03 +18) | 5′-GAU AGG UGG UAU CAA | Very strong |
| | | CAU CUG | skipping to 40 nM |
| 3 | H8A(−07 +18) | 5′-GAU AGG UGG UAU CAA | Strong skipping to |
| | | CAU CUG UAA G | 40 nM |
| 4 | H8A(−06 +14) | 5′-GGU GGU AUC AAC AUC | Skipping to |
| | | UGU AA | 300 nM |
| 5 | H8A(−10 +10) | 5′-GUA UCA ACA UCU GUA | Patchy/weak |
| | | AGC AC | skipping to 100 nm |

### Antisense Oligonucleotides Directed at Exon 7

Antisense oligonucleotides directed at exon 7 were prepared and tested for their ability to induce exon skipping in human muscle cells using similar methods as described above.

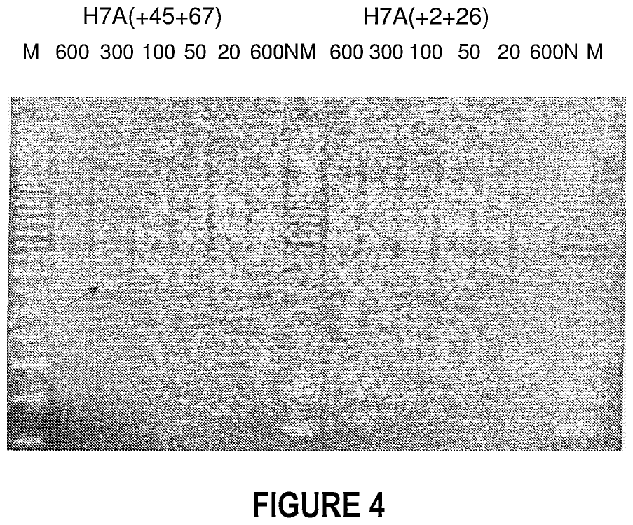

FIG. 4 shows the preferred antisense molecule, H7A(+45+67) [SEQ ID NO: 6], and another antisense molecule, H7A(+2+26) [SEQ ID NO: 7], inducing exon 7 skipping. Nested amplification products span exons 3 to 9. Additional products above the induced transcript missing exon 7 arise from amplification from carry-over outer primers from the RT-PCR as well as heteroduplex formation.

Table 3 below discloses antisense molecule sequences for induced exon 7 skipping.

TABLE 3

| | Antisense | | |
|---|---|---|---|
| SEQ | Oligonucleotide | | Ability to induce |
| ID | name | Sequence | skipping |
| 6 | H7A(+45 +67) | 5′ - UGC AUG UUC CAG UCG UUG UGU | Strong skipping |
| | | GG | to 20 nM |
| 7 | H7A(+02 +26) | 5′ - CAC UAU UCC AGU CAA AUA GGU | Weak skipping at |
| | | CUG G | 100 nM |
| 8 | H7D(+15 −10) | 5′ -AUU UAC CAA CCU UCA GGA UCG | Weak skipping to |
| | | AGU A | 300 nM |
| 9 | H7A(−18 +03) | 5′ - GGC CUA AAA CAC AUA CAC AUA | Weak skipping to |
| | | | 300 nM |

### Antisense Oligonucleotides Directed at Exon 6

Antisense oligonucleotides directed at exon 6 were prepared and tested for their ability to induce exon skipping in human muscle cells using similar methods as described above.

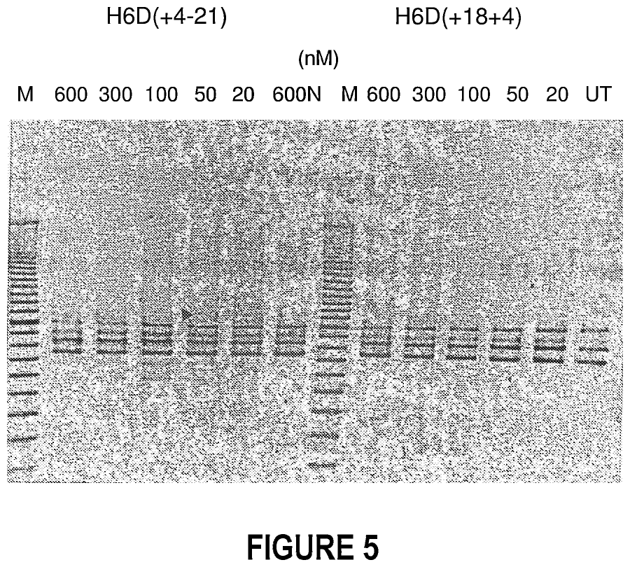

FIG. 5 shows an example of two non-preferred antisense molecules inducing very low levels of exon 6 skipping in cultured human cells. Targeting this exon for specific removal was first undertaken during a study of the canine model using the oligonucleotides as listed in Table 4, below. Some of the human specific oligonucleotides were also evaluated, as shown in FIG. 5. In this example, both antisense molecules target the donor splice site and only induced low levels of exon 6 skipping. Both H6D(+4−21) [SEQ ID NO: 17] and H6D(+18−4) [SEQ ID NO: 18] would be regarded as non-preferred antisense molecules.

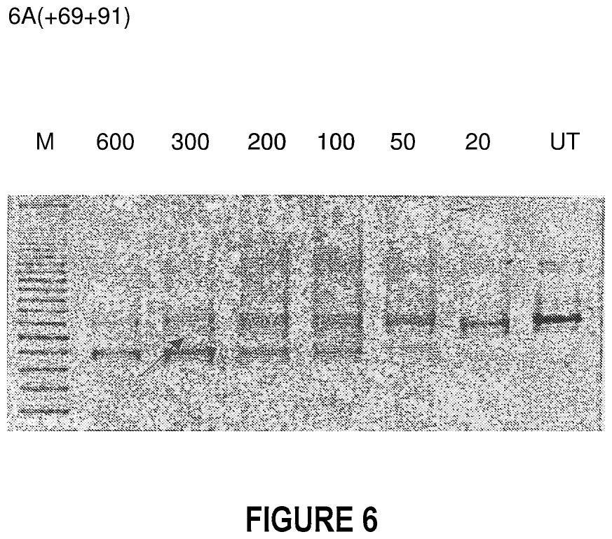

One antisense oligonucleotide that induced very efficient exon 6 skipping in the canine model, C6A(+69+91) [SEQ ID NO: 14], would anneal perfectly to the corresponding region in human dystrophin exon 6. This compound was evaluated, found to be highly efficient at inducing skipping of that target exon, as shown in FIG. 6 and is regarded as the preferred compound for induced exon 6 skipping. Table 4 below discloses antisense molecule sequences for induced exon 6 skipping.

TABLE 4

| | Antisense Oligo | | Ability to induce |
|---|---|---|---|
| SEQ ID | name | Sequence | skipping |
| 10 | C6A(−10 +10) | 5′ CAU UUU UGA CCU ACA UGU | No skipping |
| | | GG | |
| 11 | C6A(−14 +06) | 5′ UUU GAC CUA CAU GUG GAA | No skipping |
| | | AG | |
| 12 | C6A(−14+ 12) | 5′ UAC AUU UUU GAC CUA CAU | No skipping |
| | | GUG GAA AG | |
| 13 | C6A(−13 +09) | 5′ AUU UUU GAC CUA CAU GGG | No skipping |
| | | AAA G | |
| 14 | CH6A(+69 +91) | 5′ UAC GAG UUG AUU GUC GGA | Strong skipping to 20 |
| | | CCC AG | nM |
| 15 | C6D(+12 −13) | 5′ GUG GUC UCC UUA CCU AUG | Weak skipping at 300 |
| | | ACU GUG G | nM |
| 16 | C6D(+06 −11) | 5′ GGU CUC CUU ACC UAU GA | No skipping |
| 17 | H6D(+04 −21) | 5′ UGU CUC AGU AAU CUU CUU | Weak skipping to 50 |
| | | ACC UAU | nM |
| 18 | H6D(+18 −04) | 5′ UCU UAC CUA UGA CUA UGG | Very weak skipping to |
| | | AUG AGA | 300 nM |

### Antisense Oligonucleotides Directed at Exon 4

Antisense oligonucleotides directed at exon 4 were prepared and tested for their ability to induce exon skipping in human muscle cells using similar methods as described above.

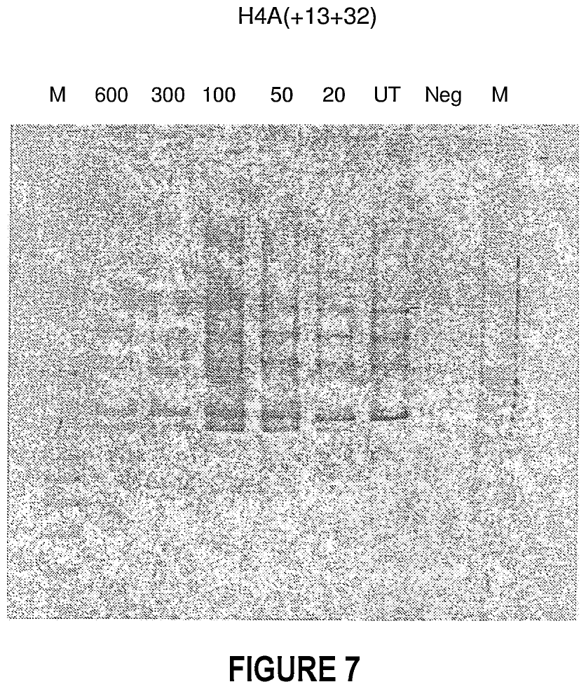

FIG. 7 shows an example of a preferred antisense molecule inducing skipping of exon 4 skipping in cultured human cells. In this example, one preferred antisense compound, H4A(+13+32) [SEQ ID NO:19], which targeted a presumed exonic splicing enhancer induced efficient exon skipping at a concentration of 20 nM while other non-preferred antisense oligonucleotides failed to induce even low levels of exon 4 skipping. Another preferred antisense molecule inducing skipping of exon 4 was H4A(+111+40) [SEQ ID NO:22], which induced efficient exon skipping at a concentration of 20 nM.

Table 5 below discloses antisense molecule sequences for inducing exon 4 skipping.

TABLE 5

| | | | Ability to |
|---|---|---|---|
| SEQ | Antisense | | induce |
| ID | Oligonucleotide name | Sequence | skipping |
| 19 | H4A(+13 +32) | 5′ GCA UGA ACU CUU GUG GAU CC | Skipping to |
| | | | 20 nM |
| 22 | H4A(+11 +40) | 5′ UGU UCA GGG CAU GAA CUC UUG UGG | Skipping to |
| | | AUC CUU | 20 nM |
| 20 | H4D(+04 −16) | 5′ CCA GGG UAC UAC UUA CAU UA | No skipping |
| 21 | H4D(−24 −44) | 5′ AUC GUG UGU CAC AGC AUC CAG | No skipping |

### Antisense Oligonucleotides Directed at Exon 3

Antisense oligonucleotides directed at exon 3 were prepared and tested for their ability to induce exon skipping in human muscle cells using similar methods as described above.

H3A(+30+60) [SEQ ID NO:23] induced substantial exon 3 skipping when delivered into cells at a concentration of 20 nM to 600 nM. The antisense molecule, H3A(+35+65) [SEQ ID NO: 24] induced exon skipping at 300 nM.

Table 6 below discloses antisense molecule sequences that induce exon 3 skipping.

TABLE 6

| | | | Ability to |
|---|---|---|---|
| | Antisense | | induce |
| SEQ ID | Oligonucleotide name | Sequence | skipping |
| 23 | H3A(+30 +60) | UAG GAG GCG CCU CCC AUC CUG UAG | Moderate |
| | | GUC ACU G | skipping to |
| | | | 20 to 600 nM |
| 24 | H3A(+35 +65) | AGG UCU AGG AGG CGC CUC CCA UCC | Working to |
| | | UGU AGG U | 300 nM |
| 25 | H3A(+30 +54) | GCG CCU CCC AUC CUG UAG GUC ACU G | Moderate |
| | | | 100-600 nM |
| 26 | H3D(+46 −21) | CUU CGA GGA GGU CUA GGA GGC GCC | No skipping |
| | | UC | |
| 27 | H3A(+30 +50) | CUC CCA UCC UGU AGG UCA CUG | Moderate 20-600 nM |
| 28 | H3D(+19 −03) | UAC CAG UUU UUG CCC UGU CAG G | No skipping |
| 29 | H3A(−06 +20) | UCA AUA UGC UGC UUCCCA AAC UGA | No skipping |
| | | AA | |
| 30 | H3A(+37 +61) | CUA GGA GGC GCC UCC CAU CCU GUA G | No skipping |

### Antisense Oligonucleotides Directed at Exon 5

Antisense oligonucleotides directed at exon 5 were prepared and tested for their ability to induce exon skipping in human muscle cells using similar methods as described above.

H5A(+20+50) [SEQ ID NO:31] induces substantial exon 5 skipping when delivered into cells at a concentration of 100 nM. Table 7 below shows other antisense molecules tested. The majority of these antisense molecules were not as effective at exon skipping as H5A(+20+50). However, H5A(+15+45) [SEQ ID NO: 40] was able to induce exon 5 skipping at 300 nM.

Table 7 below discloses antisense molecule sequences that induce exon 5 skipping.

TABLE 7

| | Antisense | | Ability to |
|---|---|---|---|
| | Oligonucleotide | Sequence | induce |
| SEQ ID | name | | skipping |
| 31 | H5A(+20 +50) | UUA UGA UUU CCA UCU ACG | Working to |
| | | AUG UCA GUA CUU C | 100 nM |
| 32 | H5D(+25 −05) | CUU ACC UGC CAG UGG AGG | No skipping |
| | | AUU AUA UUC CAA A | |
| 33 | H5D(+10 −15) | CAU CAG GAU UCU UAC CUG | Inconsistent |
| | | CCA GUG G | at 300 nM |
| 34 | H5A(+10 +34) | CGA UGU CAG UAC UUC CAA | Very weak |
| | | UAU UCA C | |
| 35 | H5D(−04 −21) | ACC AUU CAU CAG GAU UCU | No skipping |
| 36 | H5D(+16 −02) | ACC UGC CAG UGG AGG AUU | No skipping |
| 37 | H5A(−07 +20) | CCA AUA UUC ACU AAA UCA | No skipping |
| | | ACC UGU UAA | |
| 38 | H5D(+18 −12) | CAG GAU UCU UAC CUG CCA | No skipping |
| | | GUG GAG GAU UAU | |
| 39 | H5A(+05 +35) | ACG AUG UCA GUA CUU CCA | No skipping |
| | | AUA UUC ACU AAA U | |
| 40 | H5A(+15 +45) | AUU UCC AUC UAC GAU GUC | Working to |
| | | AGU ACU UCC AAU A | 300 nM |

### Antisense Oligonucleotides Directed at Exon 10

Antisense oligonucleotides directed at exon 10 were prepared and tested for their ability to induce exon skipping in human muscle cells using similar methods as described above.

H10A(−05+16) [SEQ ID NO:41] induced substantial exon 10 skipping when delivered into cells. Table 8 below shows other antisense molecules tested. The antisense molecules ability to induce exon skipping was variable. Table 8 below discloses antisense molecule sequences that induce exon 10 skipping.

TABLE 8

| SEQ | Antisense | | Ability to |
|---|---|---|---|
| ID | Oligonucleotide name | Sequence | induce skipping |
| 41 | H10A(−05 +16) | CAG GAG CUU CCA AAU GCU GCA | Not tested |
| 42 | H10A(−05 +24) | CUU GUC UUC AGG AGC UUC CAA | Not tested |
| | | AUG CUG CA | |
| 43 | H10A(+98 +119) | UCC UCA GCA GAA AGA AGC CAC G | Not tested |
| 44 | H10A(+130 +149) | UUA GAA AUC UCU CCU UGU GC | No skipping |
| 45 | H10A(−33 −14) | UAA AUU GGG UGU UAC ACA AU | No skipping |

### Antisense Oligonucleotides Directed at Exon 11

Antisense oligonucleotides directed at exon 11 were prepared and tested for their ability to induce exon skipping in human muscle cells using similar methods as described above.

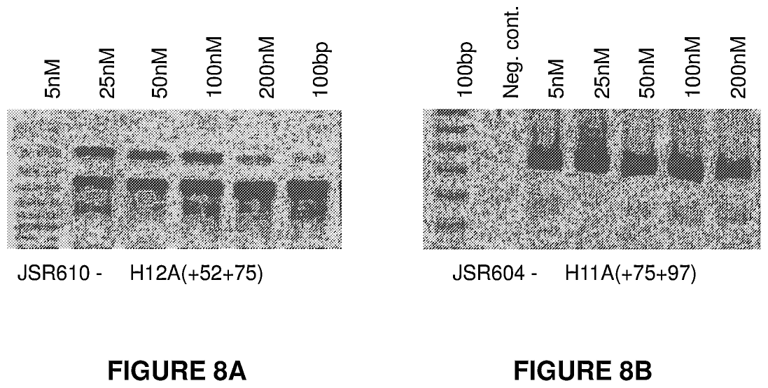

FIG. 8B shows an example of H11A(+75+97) [SEQ ID NO:49] antisense molecule inducing exon 11 skipping in cultured human cells. H11A(+75+97) induced substantial exon 11 skipping when delivered into cells at a concentration of 5 nM. Table 9 below shows other antisense molecules tested. The antisense molecules ability to induce exon skipping was observed at 100 nM.

TABLE 9

| SEQ | Antisense | | Ability to |
|---|---|---|---|
| ID | Oligonucleotide name | Sequence | induce skipping |
| 46 | H11D(+26 +49) | CCC UGA GGC AUU CCC AUC UUG | Skipping at 100 |
| | | AAU | nM |
| 47 | H11D(+11 −09) | AGG ACU UAC UUG CUU UGU UU | Skipping at 100 |
| | | | nM |
| 48 | H11A(+118 +140) | CUU GAA UUU AGG AGA UUC AUC UG | Skipping at 100 |
| | | | nM |
| 49 | H11A(+75 +97) | CAU CUU CUG AUA AUU UUC CUG UU | Skipping at 100 |
| | | | nM |
| 46 | H11D(+26 +49) | CCC UGA GGC AUU CCC AUC UUG | Skipping at |
| | | AAU | 5 nM |

### Antisense Oligonucleotides Directed at Exon 12

Antisense oligonucleotides directed at exon 12 were prepared and tested for their ability to induce exon skipping in human muscle cells using similar methods as described above.

H12A(+52+75) [SEQ ID NO:50] induced substantial exon 12 skipping when delivered into cells at a concentration of 5 nM, as shown in FIG. 8A. Table 10 below shows other antisense molecules tested at a concentration range of 5, 25, 50, 100, 200 and 300 nM. The antisense molecules ability to induce exon skipping was variable.

TABLE 10

| | Antisense | | Ability to |
|---|---|---|---|
| | Oligonucleotide | | induce |
| SEQ ID | name | Sequence | skipping |
| 50 | H12A(+52 +75) | UCU UCU GUU UUU GUU AGC CAG UCA | Skipping at 5 |
| | | | nM |
| 51 | H12A(−10 +10) | UCU AUG UAA ACU GAA AAU UU | Skipping at |
| | | | 100 nM |
| 52 | H12A(+11 +30) | UUC UGG AGA UCC AUU AAA AC | No skipping |

### Antisense Oligonucleotides Directed at Exon 13

Antisense oligonucleotides directed at exon 13 were prepared and tested for their ability to induce exon skipping in human muscle cells using similar methods as described above.

H13A(+77+100) [SEQ ID NO:53] induced substantial exon 13 skipping when delivered into cells at a concentration of 5 nM. Table 11 below includes two other antisense molecules tested at a concentration range of 5, 25, 50, 100, 200 and 300 nM. These other antisense molecules were unable to induce exon skipping.

TABLE 11

| | Antisense | | Ability to |
|---|---|---|---|
| SEQ | Oligonucleo- | | induce |
| ID | tide name | Sequence | skipping |
| 53 | H13A(+77 +100) | CAG CAG UUG CGU GAU CUC CAC UAG | Skipping at 5 |
| | | | nM |
| 54 | H13A(+55 +75) | UUC AUC AAC UAC CAC CAC CAU | No skipping |
| 55 | H13D(+06 −19) | CUA AGC AAA AUA AUC UGA CCU UAA | No skipping |
| | | G | |

### Antisense Oligonucleotides Directed at Exon 14

Antisense oligonucleotides directed at exon 14 were prepared and tested for their ability to induce exon skipping in human muscle cells using similar methods as described above.

H14A(+37+64) [SEQ ID NO:56] induced weak exon 14 skipping when delivered into cells at a concentration of 100 nM. Table 12 below includes other antisense molecules tested at a concentration range of 5, 25, 50, 100, 200 and 300 nM. The other antisense molecules were unable to induce exon skipping at any of the concentrations tested.

TABLE 12

| | Antisense | | Ability to |
|---|---|---|---|
| SEQ | Oligonucleotide | | induce |
| ID | name | Sequence | skipping |
| 56 | H14A(+37 +64) | CUU GUA AAA GAA CCC AGC | Skipping at |
| | | GGU CUU CUG U | 100 nM |
| 57 | H14A(+14 +35) | CAU CUA CAG AUG UUU GCC | No skipping |
| | | CAU C | |
| 58 | H14A(+51 +73) | GAA GGA UGU CUU GUA AAA | No skipping |
| | | GAA CC | |
| 59 | H14D(−02 +18) | ACC UGU UCU UCA GUA AGA | No skipping |
| | | CG | |
| 60 | H14D(+14 −10) | CAU GAC ACA CCU GUU CUU | No skipping |
| | | CAG UAA | |
| 61 | H14A(+61 +80) | CAU UUG AGA AGG AUG UCU | No skipping |
| | | UG | |
| 62 | H14A(−12 +12) | AUC UCC CAA UAC CUG GAG | No skipping |
| | | AAG AGA | |

### Antisense Oligonucleotides Directed at Exon 15

Antisense oligonucleotides directed at exon 15 were prepared and tested for their ability to induce exon skipping in human muscle cells using similar methods as described above.

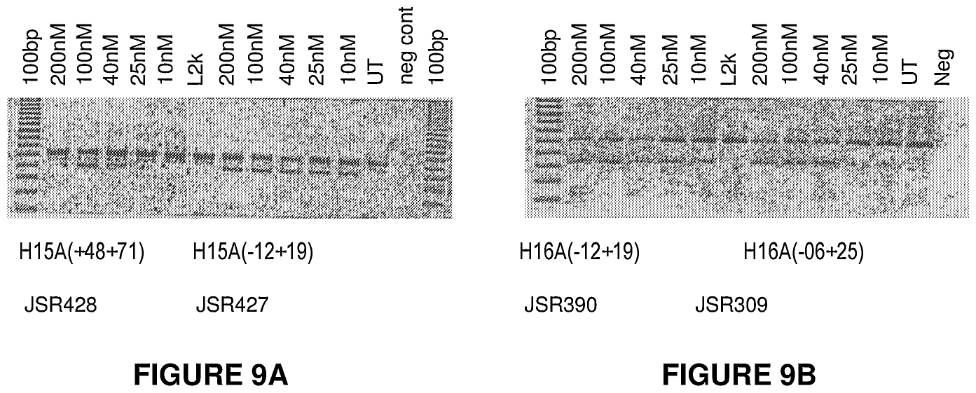

H15A(−12+19) [SEQ ID NO:63] and H15A(+48+71) [SEQ ID NO:64] induced substantial exon 15 skipping when delivered into cells at a concentration of 10 Nm, as shown in FIG. 9A. Table 13 below includes other antisense molecules tested at a concentration range of 5, 25, 50, 100, 200 and 300 Nm. These other antisense molecules were unable to induce exon skipping at any of the concentrations tested.

TABLE 13

| | Antisense | | Ability to |
|---|---|---|---|
| SEQ | Oligonucleotide | | induce |
| ID | name | Sequence | skipping |
| 63 | H15A(−12 +19) | GCC AUG CAC UAA AAA GGC ACU GCA AGA | Skipping at |
| | | CAU U | 5 Nm |
| 64 | H15A(+48 +71) | UCU UUA AAG CCA GUU GUG UGA AUC | Skipping at |
| | | | 5 Nm |
| 65 | H15A(+08 +28) | UUU CUG AAA GCC AUG CAC UAA | No skipping |
| 63 | H15A(−12 +19) | GCC AUG CAC UAA AAA GGC ACU GCA AGA | No skipping |
| | | CAU U | |
| 66 | H15D(+17 −08) | GUA CAU ACG GCC AGU UUU UGA AGA C | No skipping |

### Antisense Oligonucleotides Directed at Exon 16

Antisense oligonucleotides directed at exon 16 were prepared and tested for their ability to induce exon skipping in human muscle cells using similar methods as described above.

H16A(−12+19) [SEQ ID NO:67] and H16A(−06+25) [SEQ ID NO:68] induced substantial exon 16 skipping when delivered into cells at a concentration of 10 nM, as shown in FIG. 9B. Table 14 below includes other antisense molecules tested. H16A(−06+19) [SEQ ID NO:69] and H16A(+87+109) [SEQ ID NO:70] were tested at a concentration range of 5, 25, 50, 100, 200 and 300 nM. These two antisense molecules were able to induce exon skipping at 25 nM and 100 nM, respectively. Additional antisense molecules were tested at 100, 200 and 300 nM and did not result in any exon skipping.

TABLE 14

| | Antisense | | Ability to |
|---|---|---|---|
| SEQ | Oligonucleotide | | induce |
| ID | name | Sequence | skipping |
| 67 | H16A(−12 +19) | CUA GAU CCG CUU UUA AAA CCU GUU | Skipping at |
| | | AAA ACA A | 5 nM |
| 68 | H16A(−06 +25) | UCU UUU CUA GAU CCG CUU UUA AAA | Skipping at |
| | | CCU GUU A | 5 nM |
| 69 | H16A(−06 +19) | CUA GAU CCG CUU UUA AAA CCU GUU A | Skipping at |
| | | | 25 nM |
| 70 | H16A(+87 +109) | CCG UCU UCU GGG UCA CUG ACU UA | Skipping at |
| | | | 100 nM |
| 71 | H16A(−07 +19) | CUA GAU CCG CUU UUA AAA CCU GUU AA | No skipping |
| 72 | H16A(−07 +13) | CCG CUU UUA AAA CCU GUU AA | No skipping |
| 73 | H16A(+12 +37) | UGG AUU GCU UUU UCU UUU CUA GAU CC | No skipping |
| 74 | H16A(+92 +116) | CAU GCU UCC GUC UUC UGG GUC ACU G | No skipping |
| 75 | H16A(+45 +67) | G AUC UUG UUU GAG UGA AUA CAG U | No skipping |
| 76 | H16A(+105 +126) | GUU AUC CAG CCA UGC UUC CGU C | No skipping |
| 77 | H16D(+05 −20) | UGA UAA UUG GUA UCA CUA ACC UGU G | No skipping |
| 78 | H16D(+12 −11) | GUA UCA CUA ACC UGU GCU GUA C | No skipping |

### Antisense Oligonucleotides Directed at Exon 19

Antisense oligonucleotides directed at exon 19 were prepared and tested for their ability to induce exon skipping in human muscle cells using similar methods as described above.

H19A(+35+65) [SEQ ID NO:79] induced substantial exon 19 skipping when delivered into cells at a concentration of 10 nM. This antisense molecule also showed very strong exon skipping at concentrations of 25, 50, 100, 300 and 600 nM.

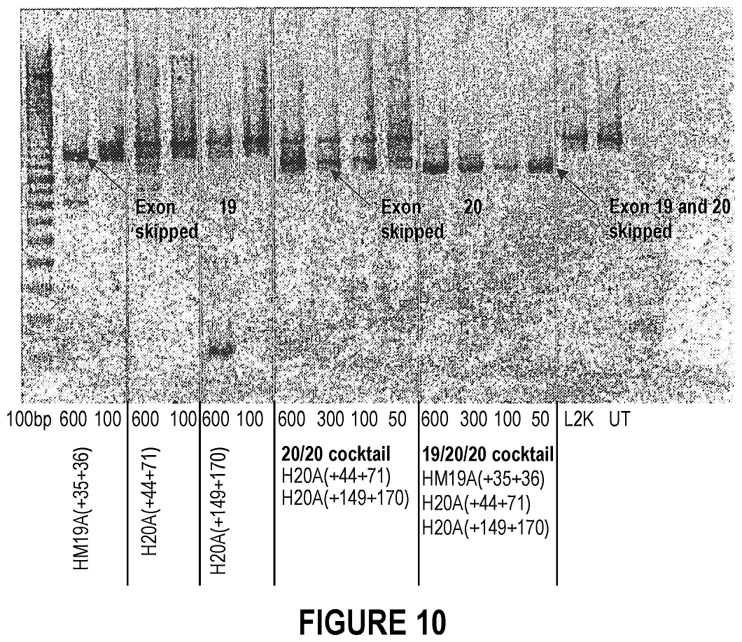

FIG. 10 illustrates exon 19 and 20 skipping using a “cocktail” of antisense oligonucleotides, as tested using gel electrophoresis. It is interesting to note that it was not easy to induce exon 20 skipping using single antisense oligonucleotides H20A(+44+71) [SEQ ID NO:81] or H20A(+149+170) [SEQ ID NO:82], as illustrated in sections 2 and 3of the gel shown in FIG. 10. Whereas, a “cocktail” of antisense oligonucleotides was more efficient as can be seen in section 4 of FIG. 10 using a “cocktail” of antisense oligonucleotides H20A(+44+71) and H20A(+149+170). When the cocktail was used to target exon 19, skipping was even stronger (see section 5, FIG. 10).

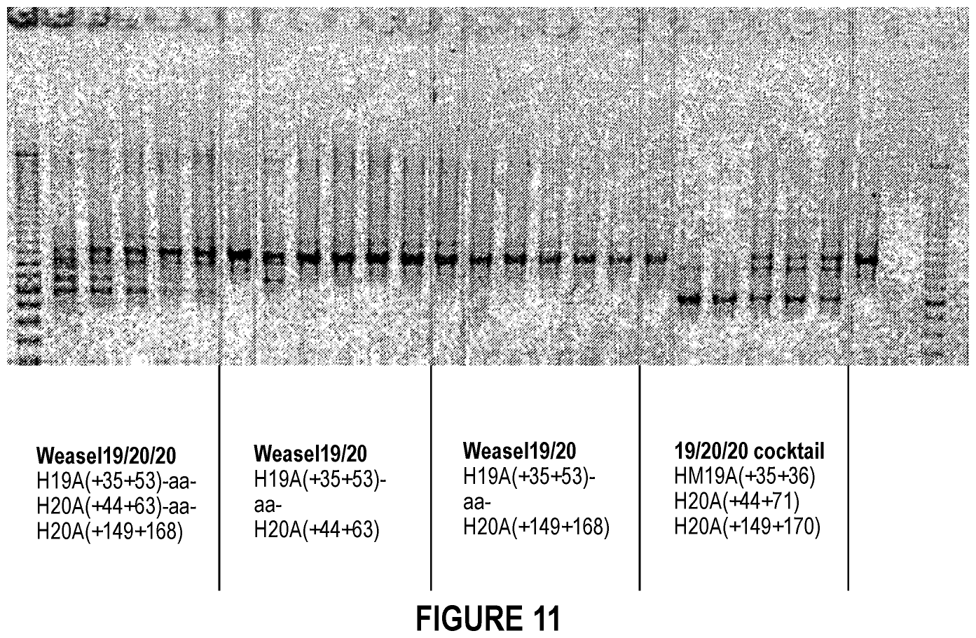

FIG. 11 illustrates gel electrophoresis results of exon 19/20 skipping using “weasels” The “weasels” were effective in skipping exons 19 and 20 at concentrations of 25, 50, 100, 300 and 600 nM. A further “weasel” sequence is shown in the last row of Table 3C. This compound should give good results.

### Antisense Oligonucleotides Directed at Exon 20

Antisense oligonucleotides directed at exon 20 were prepared and tested for their ability to induce exon skipping in human muscle cells using similar methods as described above.

None of the antisense oligonucleotides tested induced exon 20 skipping when delivered into cells at a concentration of 10, 25, 50, 300 or 600 nM (see Table 15). Antisense molecules H20A(−11+17) [SEQ ID NO:86] and H20D(+08−20) [SEQ ID NO:87] are yet to be tested.

However, a combination or “cocktail” of H20A(+44+71) [SEQ ID NO: 81] and H20(+149+170) [SEQ ID NO:82] in a ratio of 1:1, exhibited very strong exon skipping at a concentration of 100 nM and 600 nM. Further, a combination of antisense molecules H19A(+35+65) [SEQ ID NO:79], H20A(+44+71) [SEQ ID NO:81] and H20A(+149+170) [SEQ ID NO:82] in a ratio of 2:1:1, induced very strong exon skipping at a concentration ranging from 10 nM to 600 nM.

TABLE 15

| | Antisense | | Ability to |
|---|---|---|---|
| SEQ | Oligonucleotide | | induce |
| ID | name | Sequence | skipping |
| 81 | H20A(+44 +71) | CUG GCA GAA UUC GAU CCA CCG GCU | No |
| | | GUU C | skipping |
| 82 | H20A(+147 +168) | CAG CAG UAG UUG UCA UCU GCU C | No |
| | | | skipping |
| 83 | H20A(+185 +203) | UGA UGG GGU GGU GGG UUG G | No |
| | | | skipping |
| 84 | H20A(−08 +17) | AUC UGC AUU AAC ACC CUC UAG AAA G | No |
| | | | skipping |
| 85 | H20A(+30 +53) | CCG GCU GUU CAG UUG UUC UGA GGC | No |
| | | | skipping |
| 86 | H20A(−11 +17) | AUC UGC AUU AAC ACC CUC UAG AAA | Not tested |
| | | GAA A | yet |
| 87 | H20D(+08 −20) | GAA GGA GAA GAG AUU CUU ACC UUA | Not tested |
| | | CAA A | yet |
| 81 & | H20A(+44 +71) & | CUG GCA GAA UUC GAU CCA CCG GCU | Very strong |
| 82 | H20A(+147 +168) | GUU C | skipping |
| | | CAG CAG UAG UUG UCA UCU GCU C | |
| 80, 81 | H19A(+35 +65); | GCC UGA GCU GAU CUG CUG GCA UCU | Very strong |
| & 82 | H20A(+44 +71); | UGC AGU U; | skipping |
| | H20A(+147 +168) | CUG GCA GAA UUC GAU CCA CCG GCU | |
| | | GUU C; | |
| | | CAG CAG UAG UUG UCA UCU GCU C | |

### Antisense Oligonucleotides Directed at Exon 21

Antisense oligonucleotides directed at exon 21 were prepared and tested for their ability to induce exon skipping in human muscle cells using similar methods as described above.

H21A(+85+108) [SEQ ID NO:92] and H21A(+85+106) [SEQ ID NO:91] induced exon 21 skipping when delivered into cells at a concentration of 50 nM. Table 16 below includes other antisense molecules tested at a concentration range of 5, 25, 50, 100, 200 and 300 nM. These antisense molecules showed a variable ability to induce exon skipping

TABLE 16

| SEQ | Antisense | | Ability to induce |
|---|---|---|---|
| ID | Oligonucleotide name | Sequence | skipping |
| 90 | H21A(−06 +16) | GCC GGU UGA CUU CAU CCU GUG C | Skips at 600 nM |
| 91 | H21A(+85 +106) | CUG CAU CCA GGA ACA UGG GUC C | Skips at 50 nM |
| 92 | H21A(+85 +108) | GUC UGC AUC CAG GAA CAU GGG | Skips at 50 nM |
| | | UC | |
| 93 | H21A(+08 +31) | GUU GAA GAU CUG AUA GCC GGU | Skips faintly to |
| | | UGA | |
| 94 | H21D(+18 −07) | UAC UUA CUG UCU GUA GCU CUU | No skipping |
| | | UCU | |

### Antisense Oligonucleotides Directed at Exon 22

Antisense oligonucleotides directed at exon 22 were prepared and tested for their ability to induce exon skipping in human muscle cells using similar methods as described above.

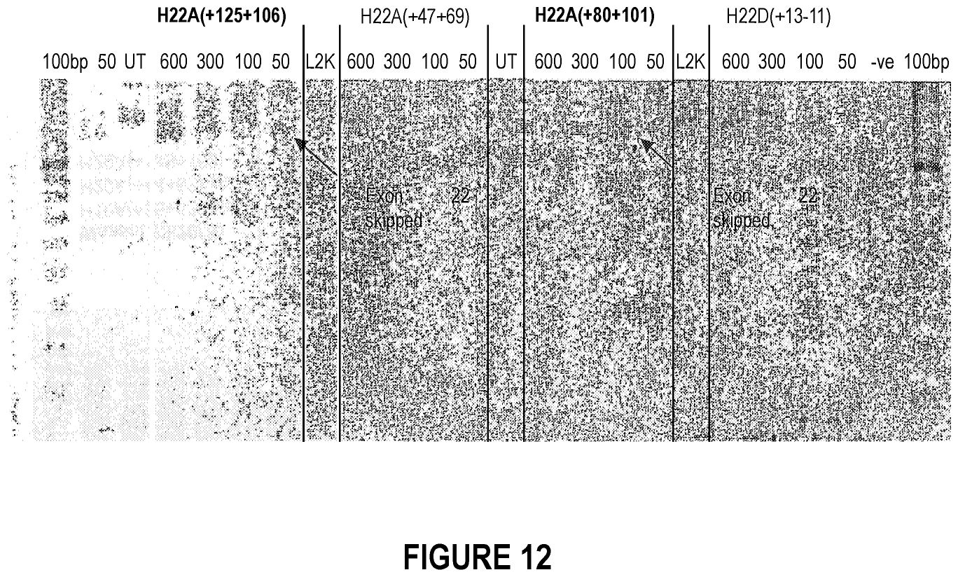

FIG. 12 illustrates differing efficiencies of two antisense molecules directed at exon 22 acceptor splice site. H22A(+125+106) [SEQ ID NO:96] and H22A(+80+101) [SEQ ID NO: 98] induce strong exon 22 skipping from 50 nM to 600 nM concentration.

H22A(+125+146) [SEQ ID NO:96] and H22A(+80+101) [SEQ ID NO:98] induced exon 22 skipping when delivered into cells at a concentration of 50 nM. Table 17 below shows other antisense molecules tested at a concentration range of 50, 100, 300 and 600 nM. These antisense molecules showed a variable ability to induce exon skipping.

TABLE 17

| | Antisense | | |
|---|---|---|---|
| SEQ | oligonucleotide | | Ability to induce |
| ID | name | Sequence | skipping |
| 95 | H22A(+22 +45) | CAC UCA UGG UCU CCU GAU AGC | No skipping |
| | | GCA | |
| 96 | H22A(+125 +146) | CUG CAA UUC CCC GAG UCU CUG | Skipping to 50 nM |
| | | C | |
| 97 | H22A(+47 +69) | ACU GCU GGA CCC AUG UCC UGA | Skipping to 300 nM |
| | | UG | |
| 98 | H22A(+80 +101) | CUA AGU UGA GGU AUG GAG AGU | Skipping to 50 nM |
| 99 | H22D(+13 −11) | UAU UCA CAG ACC UGC AAU UCC | No skipping |
| | | CC | |

### Antisense Oligonucleotides Directed at Exon 23

Antisense oligonucleotides directed at exon 23 were prepared and tested for their ability to induce exon skipping in human muscle cells using similar methods as described above.

Table 18 below shows antisense molecules tested at a concentration range of 25, 50, 100, 300 and 600 nM. These antisense molecules showed no ability to induce exon skipping or are yet to be tested.

TABLE 18

| | Antisense | | Ability to |
|---|---|---|---|
| SEQ | oligonucleotide | | induce |
| ID | name | Sequence | skipping |
| 100 | H23A(+34 +59) | ACA GUG GUG CUG AGA UAG UAU AGG | No skipping |
| | | CC | |
| 101 | H23A(+18 +39) | UAG GCC ACU UUG UUG CUC UUG C | No Skipping |
| 102 | H23A(+72 +90) | UUC AGA GGG CGC UUU CUU C | No Skipping |

### Antisense Oligonucleotides Directed at Exon 24

Antisense oligonucleotides directed at exon 24 were prepared using similar methods as described above. Table 19 below outlines the antisense oligonucleotides directed at exon 24 that are yet to be tested for their ability to induce exon 24 skipping.

TABLE 19

| | Antisense | | Ability to |
|---|---|---|---|
| SEQ | oligonucleotide | | induce |
| ID | name | Sequence | skipping |
| 103 | H24A(+48 +70) | GGG CAG GCC AUU CCU CCU UCA GA | Needs testing |
| 104 | H24A(−02 +22) | UCU UCA GGG UUU GUA UGU GAU | Needs testing |
| | | UCU | |

### Antisense Oligonucleotides Directed at Exon 25

Antisense oligonucleotides directed at exon 25 were prepared using similar methods as described above. Table 20 below shows the antisense oligonucleotides directed at exon 25 that are yet to be tested for their ability to induce exon 25 skipping.

TABLE 20

| | Antisense | | Ability to |
|---|---|---|---|
| SEQ | oligonucleotide | | induce |
| ID | name | Sequence | skipping |
| 105 | H25A(+9 +36) | CUG GGC UGA AUU GUC UGA AUA | Needs testing |
| | | UCA CUG | |
| 106 | H25A(+131 +156) | CUG UUG GCA CAU GUG AUC CCA CUG | Needs testing |
| | | AG | |
| 107 | H25D(+16 −08) | GUC UAU ACC UGU UGG CAC AUG UGA | Needs testing |

### Antisense Oligonucleotides Directed at Exon 26

Antisense oligonucleotides directed at exon 26 were prepared using similar methods as described above. Table 21 below outlines the antisense oligonucleotides directed at exon 26 that are yet to be tested for their ability to induce exon 26 skipping.

TABLE 21

| | Antisense | | Ability to |
|---|---|---|---|
| SEQ | oligonucleotide | | induce |
| ID | name | Sequence | skipping |
| 108 | H26A(+132 +156) | UGC UUU CUG UAA UUC AUC UGG AGU | Needs testing |
| | | U | |
| 109 | H26A(−07 +19) | CCU CCU UUC UGG CAU AGA CCU UCC | Needs testing |
| | | AC | |
| 110 | H26A(+68 +92) | UGU GUC AUC CAU UCG UGC AUC UCU | Faint skipping |
| | | G | at 600 nM |

### Antisense Olioonucleotides Directed at Exon 27

Antisense oligonucleotides directed at exon 27 were prepared using similar methods as described above. Table 22 below outlines the antisense oligonucleotides directed at exon 27 that are yet to be tested for their ability to induce exon 27 skipping.

TABLE 22

| | Antisense | | Ability to induce |
|---|---|---|---|
| SEQ | oligonucleotide | | skipping |
| ID | name | Sequence | |
| 111 | H27A(+82 +106) | UUA AGG CCU CUU GUG CUA CAG | Needs testing |
| | | GUG G | |
| 112 | H27A(−4 +19) | GGG CCU CUU CUU UAG CUC UCU | Faint skipping at |
| | | GA | 600 and 300 nM |
| 113 | H27D(+19 −03) | GAC UUC CAA AGU CUU GCA UUU C | v. strong skipping |
| | | | at 600 and 300 nM |

### Antisense Oligonucleotides Directed at Exon 28

Antisense oligonucleotides directed at exon 28 were prepared using similar methods as described above. Table 23 below outlines the antisense oligonucleotides directed at exon 28 that are yet to be tested for their ability to induce exon 28 skipping.

TABLE 23

| | Antisense | | |
|---|---|---|---|
| SEQ | oligonucleotide | | Ability to induce |
| ID | name | Sequence | skipping |
| 114 | H28A(−05 +19) | GCC AAC AUG CCC AAA CUU CCU | v. strong skipping |
| | | AAG | at 600 and 300 |
| | | | nM |
| 115 | H28A(+99 +124) | CAG AGA UUU CCU CAG CUC CGC | Needs testing |
| | | CAG GA | |
| 116 | H28D(+16 −05) | CUU ACA UCU AGC ACC UCA GAG | v. strong skipping |
| | | | at 600 and 300 |
| | | | nM |

### Antisense Oligonucleotides Directed at Exon 29

Antisense oligonucleotides directed at exon 29 were prepared using similar methods as described above. Table 24 below outlines the antisense oligonucleotides directed at exon 29 that are yet to be tested for their ability to induce exon 29 skipping.

TABLE 24

| | Antisense | | |
|---|---|---|---|
| SEQ | oligonucleotide | | Ability to |
| ID | name | Sequence | induce skipping |
| 117 | H29A(+57 +81) | UCC GCC AUC UGU UAG GGU CUG | Needs testing |
| | | UGC C | |
| 118 | H29A(+18 +42) | AUU UGG GUU AUC CUC UGA AUG | v. strong skipping |
| | | UCG C | at 600 and 300 nM |
| 119 | H29D(+17 −05) | CAU ACC UCU UCA UGU AGU UCC | v. strong skipping |
| | | C | at 600 and 300 nM |

### Antisense Oligonucleotides Directed at Exon 30

Antisense oligonucleotides directed at exon 30 were prepared using similar methods as described above. Table 25 below outlines the antisense oligonucleotides directed at exon 30 that are yet to be tested for their ability to induce exon 30 skipping.

TABLE 25

| | Antisense | | |
|---|---|---|---|
| | oligonucleotide | | Ability to |
| SEQ ID | name | Sequence | induce skipping |
| 120 | H30A(+122 +147) | CAU UUG AGC UGC GUC CAC | Needs testing |
| | | CUU GUC UG | |
| 121 | H30A(+25 +50) | UCC UGG GCA GAC UGG AUG | Very strong skipping at |
| | | CUC UGU UC | 600 and 300 nM. |
| 122 | H30D(+19 −04) | UUG CCU GGG CUU CCU GAG | Very strong skipping at |
| | | GCA UU | 600 and 300 nM. |

### Antisense Oligonucleotides Directed at Exon 31

Antisense oligonucleotides directed at exon 31 were prepared and tested for their ability to induce exon skipping in human muscle cells using similar methods as described above.

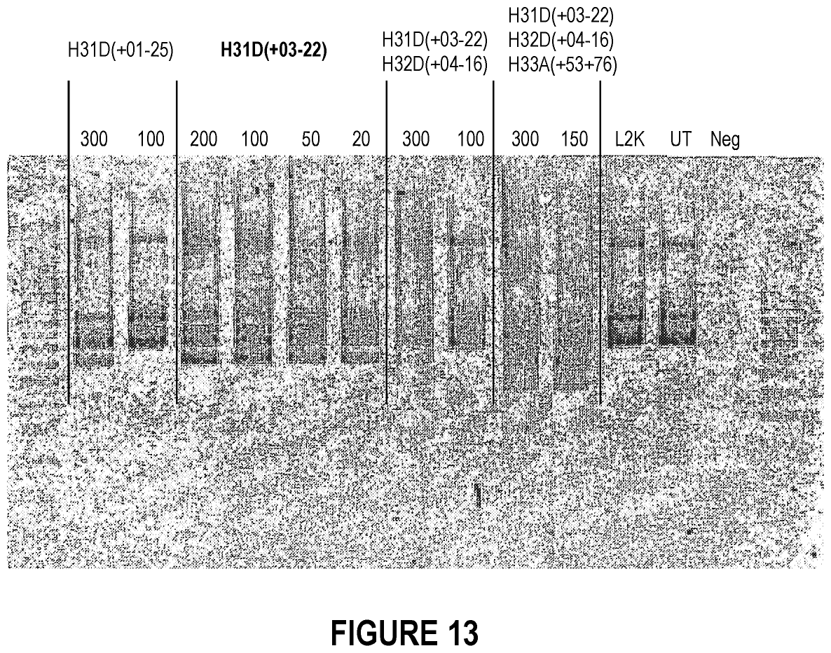

FIG. 13 illustrates differing efficiencies of two antisense molecules directed at exon 31 acceptor splice site and a “cocktail” of exon 31 antisense oligonucleotides at varying concentrations. H31D(+03−22) [SEQ ID NO: 124] substantially induced exon 31 skipping when delivered into cells at a concentration of 20 nM. Table 26 below also includes other antisense molecules tested at a concentration of 100 and 300 nM. These antisense molecules showed a variable ability to induce exon skipping.

TABLE 26

| | Antisense | | |
|---|---|---|---|
| | oligonucleotide | | Ability to |
| SEQ ID | name | Sequence | induce skipping |
| 123 | H31D(+06 −18) | UUC UGA AAU AAC AUA UAC CUG | Skipping to 300 nM |
| | | UGC | |
| 124 | H31D(+03 −22) | UAG UUU CUG AAA UAA CAU AUA | Skipping to 20 nM |
| | | CCU G | |
| 125 | H31A(+05 +25) | GAC UUG UCA AAU CAG AUU GGA | No skipping |
| 126 | H31D(+04 −20) | GUU UCU GAA AUA ACA UAU ACC | Skipping to 300 nM |
| | | UGU | |

### Antisense Oligonucleotides Directed at Exon 32

Antisense oligonucleotides directed at exon 32 were prepared and tested for their ability to induce exon skipping in human muscle cells using similar methods as described above.

H32D(+04−16) [SEQ ID NO: 127] and H32A(+49+73) [SEQ ID NO:130] induced exon 32 skipping when delivered into cells at a concentration of 300 nM. Table 27 below also shows other antisense molecules tested at a concentration of 100 and 300 nM. These antisense molecules did not show an ability to induce exon skipping.

TABLE 27

| | Antisense | | |
|---|---|---|---|
| | oligonucleotide | | Ability to |
| SEQ ID | name | Sequence | induce skipping |
| 127 | H32D(+04 −16) | CAC CAG AAA UAC AUA CCA CA | Skipping to 300 nM |
| 128 | H32A(+151 +170) | CAA UGA UUU AGC UGU GAC UG | No skipping |
| 129 | H32A(+10 +32) | CGA AAC UUC AUG GAG ACA UCU | No skipping |
| | | UG | |
| 130 | H32A(+49 +73) | CUU GUA GAC GCU GCU CAA AAU | Skipping to 300 nM |
| | | UGG C | |

### Antisense Oligonucleotides Directed at Exon 33

Antisense oligonucleotides directed at exon 33 were prepared and tested for their ability to induce exon skipping in human muscle cells using similar methods as described above.

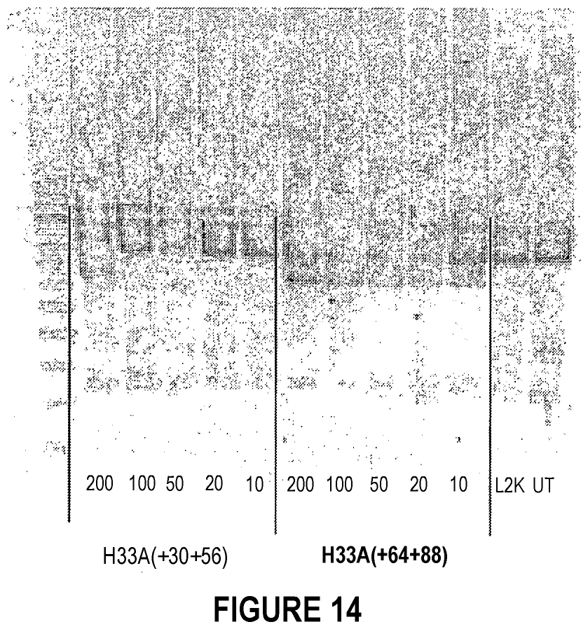

FIG. 14 shows differing efficiencies of two antisense molecules directed at exon 33 acceptor splice site. H33A(+64+88) [SEQ ID NO:134] substantially induced exon 33 skipping when delivered into cells at a concentration of 10 nM. Table 28 below includes other antisense molecules tested at a concentration of 100, 200 and 300 nM. These antisense molecules showed a variable ability to induce exon skipping.

TABLE 28

| | Antisense | | |
|---|---|---|---|
| | oligonucleotide | | Ability to |
| SEQ ID | name | Sequence | induce skipping |
| 131 | H33D(+09 −11) | CAU GCA CAC ACC UUU GCU CC | No skipping |
| 132 | H33A(+53 +76) | UCU GUA CAA UCU GAC GUC CAG | Skipping to 200 nM |
| | | UCU | |
| 133 | H33A(+30 +56) | GUG UUU AUC ACC AUU UCC ACU | Skipping to 200 nM |
| | | UCA GAC | |
| 134 | H33A(+64 +88) | GCG UCU GCU UUU UCU GUA CAA | Skipping to 10 nM |
| | | UCU G | |

### Antisense Oligonucleotides Directed at Exon 34

Antisense oligonucleotides directed at exon 34 were prepared and tested for their ability to induce exon skipping in human muscle cells using similar methods as described above.

Table 29 below includes antisense molecules tested at a concentration of 100 and 300 nM. These antisense molecules showed a variable ability to induce exon skipping.

TABLE 29

| | Antisense | | |
|---|---|---|---|
| | oligonucleotide | | Ability to |
| SEQ ID | name | Sequence | induce skipping |
| 135 | H34A(+83 +104) | UCC AUA UCU GUA GCU GGC | No skipping |
| | | AGC C | |
| 136 | H34A(+143 +165) | CCA GGC AAC UUC AGA AUC | No skipping |
| | | CAA AU | |
| 137 | H34A(−20 +10) | UUU CUG UUA CCU GAA AAG | Not tested |
| | | AAU UAU AAU GAA | |
| 138 | H34A(+46 +70) | CAU UCA UUU CCU UUC GCA | Skipping to 300 nM |
| | | UCU UAC G | |
| 139 | H34A(+95 +120) | UGA UCU CUU UGU CAA UUC | Skipping to 300 nM |
| | | CAU AUC UG | |
| 140 | H34D(+10 −20) | UUC AGU GAU AUA GGU UUU | Not tested |
| | | ACC UUU CCC CAG | |
| 141 | H34A(+72 +96) | CUG UAG CUG CCA GCC AUU | No skipping |
| | | CUG UCA AG | |

### Antisense Oligonucleotides Directed at Exon 35

Antisense oligonucleotides directed at exon 35 were prepared and tested for their ability to induce exon skipping in human muscle cells using similar methods as described above.

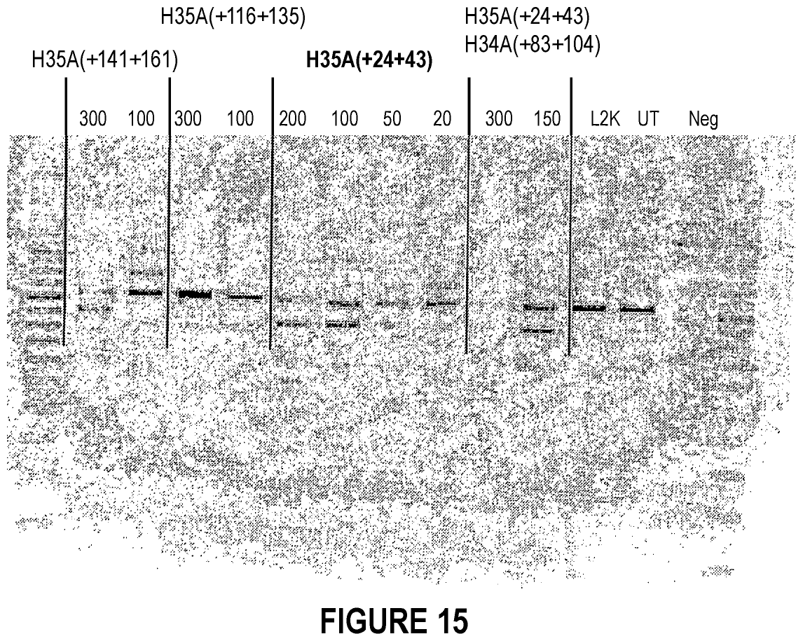

FIG. 15 shows differing efficiencies of antisense molecules directed at exon 35 acceptor splice site. H35A(+24+43) [SEQ ID NO:144] substantially induced exon 35 skipping when delivered into cells at a concentration of 20 nM. Table 30 below also includes other antisense molecules tested at a concentration of 100 and 300 nM. These antisense molecules showed no ability to induce exon skipping.

TABLE 30

| | Antisense | | |
|---|---|---|---|
| | oligo- | | Ability |
| SEQ | nucleo- | | to induce |
| ID | tide name | Sequence | skipping |
| 142 | H35A | UCU UCU GCU CGG GAG GUG ACA | Skipping |
| | (+141 | | to 20 nM |
| | +161) | | |
| 143 | H35A | CCA GUU ACU AUU CAG AAG AC | No |
| | (+116 | | skipping |
| | +135) | | |
| 144 | H35A | UCU UCA GGU GCA CCU UCU GU | No |
| | (+24 +43) | | skipping |

### Antisense Oligonucleotides Directed at Exon 36

Antisense oligonucleotides directed at exon 36 were prepared and tested for their ability to induce exon skipping in human muscle cells using similar methods as described above.

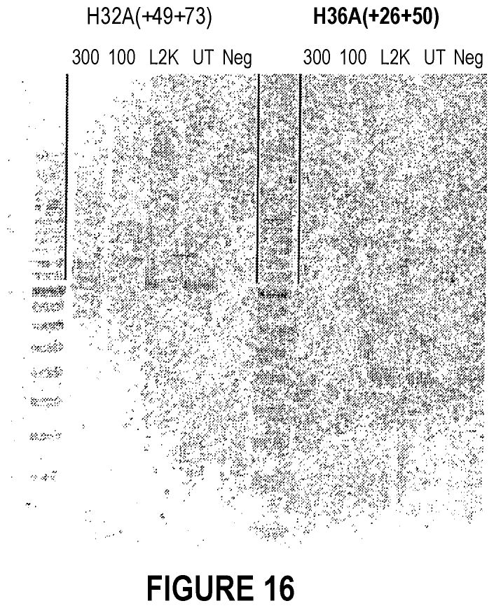

Antisense molecule H36A(+26+50) [SEQ ID NO:145] induced exon 36 skipping when delivered into cells at a concentration of 300 nM, as shown in FIG. 16.

### Antisense Oligonucleotides Directed at Exon 37

Antisense oligonucleotides directed at exon 37 were prepared and tested for their ability to induce exon skipping in human muscle cells using similar methods as described above.

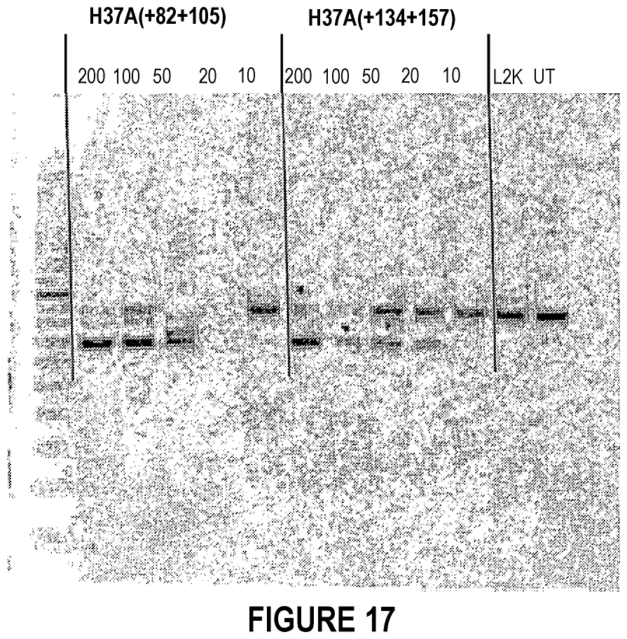

FIG. 17 shows differing efficiencies of two antisense molecules directed at exon 37 acceptor splice site. H37A(+82+105) [SEQ ID NO:148] and H37A(+134+157) [SEQ ID NO:149] substantially induced exon 37 skipping when delivered into cells at a concentration of 10 nM. Table 31 below shows the antisense molecules tested.

TABLE 31

| | Antisense | | |
|---|---|---|---|
| | oligonucleotide | | Ability to |
| SEQ ID | name | Sequence | induce skipping |
| 147 | H37A(+26 +50) | CGU GUA GAG UCC ACC UUU GGG CGU | No skipping |
| | | A | |
| 148 | H37A(+82 +105) | UAC UAA UUU CCU GCA GUG GUC ACC | Skipping to 10 nM |
| 149 | H37A(+134 +157) | UUC UGU GUG AAA UGG CUG CAA AUC | Skipping to 10 nM |

### Antisense Oligonucleotides Directed at Exon 38

Antisense oligonucleotides directed at exon 38 were prepared and tested for their ability to induce exon skipping in human muscle cells using similar methods as described above.

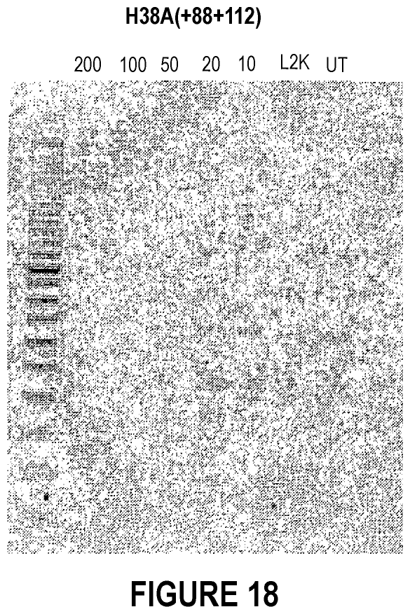

FIG. 18 illustrates antisense molecule H38A(+88+112) [SEQ ID NO:152], directed at exon 38 acceptor splice site. H38A(+88+112) substantially induced exon 38 skipping when delivered into cells at a concentration of 10 nM. Table 32 below shows the antisense molecules tested and their ability to induce exon skipping.

TABLE 32

| | Antisense | | |
|---|---|---|---|
| | oligonucleotide | | Ability to |
| SEQ ID | name | Sequence | induce skipping |
| 150 | H38A(−01 +19) | CCU UCA AAG GAA UGG AGG CC | No skipping |
| 151 | H38A(+59 +83) | UGC UGA AUU UCA GCC UCC AGU | Skipping to 10 nM |
| | | GGU U | |
| 152 | H38A(+88 +112) | UGA AGU CUU CCU CUU UCA GAU | Skipping to 10 nM |
| | | UCA C | |

### Antisense Oligonucleotides Directed at Exon 39

Antisense oligonucleotides directed at exon 39 were prepared and tested for their ability to induce exon skipping in human muscle cells using similar methods as described above.

H39A(+62+85) [SEQ ID NO:153] induced exon 39 skipping when delivered into cells at a concentration of 100 nM. Table 33 below shows the antisense molecules tested and their ability to induce exon skipping.

TABLE 33

| | Antisense | | |
|---|---|---|---|
| | oligonucleotide | | Ability to |
| SEQ ID | name | Sequence | induce skipping |
| 153 | H39A(+62 +85) | CUG GCU UUC UCU CAU CUG UGA | Skipping to 100 nM |
| | | UUC | |
| 154 | H39A(+39 +58) | GUU GUA AGU UGU CUC CUC UU | No skipping |
| 155 | H39A(+102 +121) | UUG UCU GUA ACA GCU GCU GU | No skipping |
| 156 | H39D(+10 −10) | GCU CUA AUA CCU UGA GAG CA | Skipping to 300 nM |

### Antisense Oligonucleotides Directed at Exon 40

Antisense oligonucleotides directed at exon 40 were prepared and tested for their ability to induce exon skipping in human muscle cells using similar methods as described above.

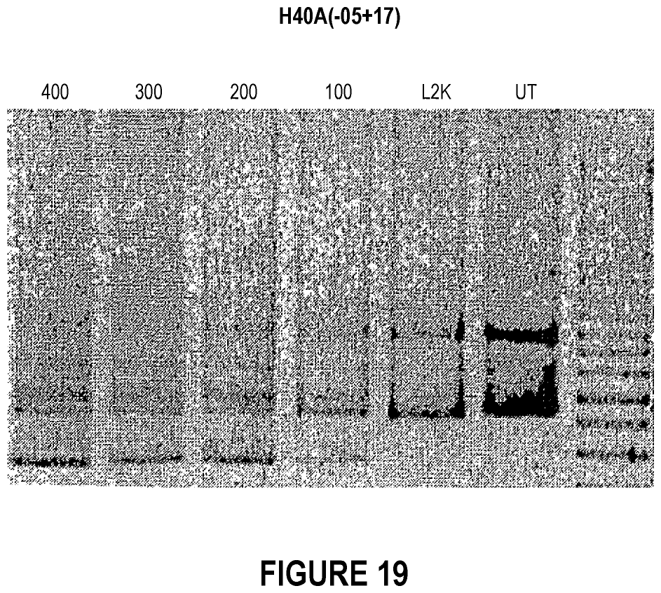

FIG. 19 illustrates antisense molecule H40A(−05+17) [SEQ ID NO:157] directed at exon 40 acceptor splice site. H40A(−05+17) and H40A(+129+153) [SEQ ID NO: 158] both substantially induced exon 40 skipping when delivered into cells at a concentration of 5 nM.

### Antisense Oligonucleotides Directed at Exon 42

Antisense oligonucleotides directed at exon 42 were prepared and tested for their ability to induce exon skipping in human muscle cells using similar methods as described above.

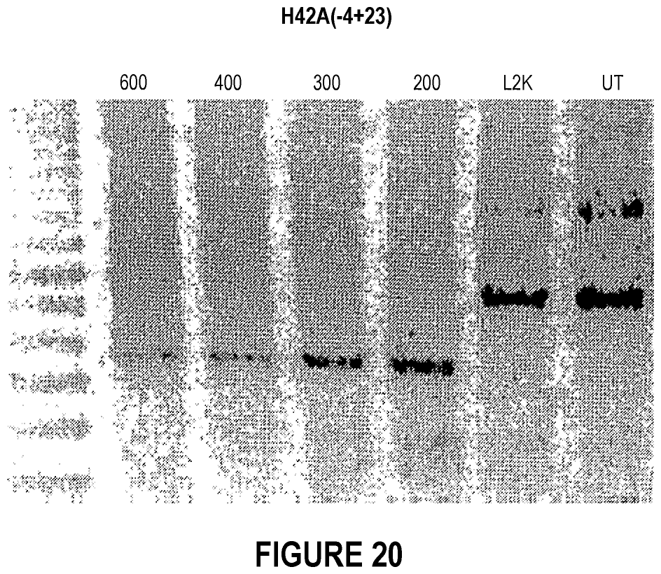

FIG. 20 illustrates antisense molecule H42A(−04+23) [SEQ ID NO:159], directed at exon 42 acceptor splice site. H42A(−4+23) and H42D(+19−02) [SEQ ID 20 NO: 161] both induced exon 42 skipping when delivered into cells at a concentration of 5 nM. Table 34 below shows the antisense molecules tested and their ability to induce exon 42 skipping.

TABLE 34

| | Antisense | | |
|---|---|---|---|
| | oligonucleotide | | Ability to |
| SEQ ID | name | Sequence | induce skipping |
| 159 | H42A(−4 +23) | AUC GUU UCU UCA CGG ACA GUG | Skipping to 5 nM |
| | | UGG UGC | |
| 160 | H42A(+86 +109) | GGG CUU GUG AGA CAU GAG UGA | Skipping to 100 nM |
| | | UUU | |
| 161 | H42D(+19 −02) | A CCU UCA GAG GAC UCC UCU | Skipping to 5 nM |
| | | UGC | |

### Antisense Oligonucleotides Directed at Exon 43

Antisense oligonucleotides directed at exon 43 were prepared and tested for their ability to induce exon skipping in human muscle cells using similar methods as described above.

H43A(+101+120) [SEQ ID NO:163] induced exon 43 skipping when delivered into cells at a concentration of 25 nM. Table 35 below includes the antisense molecules tested and their ability to induce exon 43 skipping.

TABLE 35

| | Antisense | | |
|---|---|---|---|
| | oligonucleotide | | Ability to |
| SEQ ID | name | Sequence | induce skipping |
| 162 | H43D(+10 −15) | UAU GUG UUA CCU ACC CUU GUC | Skipping to 100 nM |
| | | GGU C | |
| 163 | H43A(+101 +120) | GGA GAG AGC UUC CUG UAG CU | Skipping to 25 nM |
| 164 | H43A(+78 +100) | UCA CCC UUU CCA CAG GCG UUG CA | Skipping to 200 nM |

### Antisense Oligonucleotides Directed at Exon 44

Antisense oligonucleotides directed at exon 44 were prepared using similar methods as described above. Testing fior the ability of these antisense molecules to induce exon 44 skipping is still in progress. The antisense molecules under review are shown as SEQ ID Nos: 165 to 167 in Table 1A.

### Antisense Oligonucleotides Directed at Exon 45

Antisense oligonucleotides directed at exon 45 were prepared using similar methods as described above. Testing for the ability of these antisense molecules to induce exon 45 skipping is still in progress. The antisense molecules under review are shown as SEQ ID Nos: 207 to 211 in Table 1A.

### Antisense Oligonucleotides Directed at Exon 46

Antisense oligonucleotides directed at exon 46 were prepared and tested for their ability to induce exon skipping in human muscle cells using similar methods as described above.

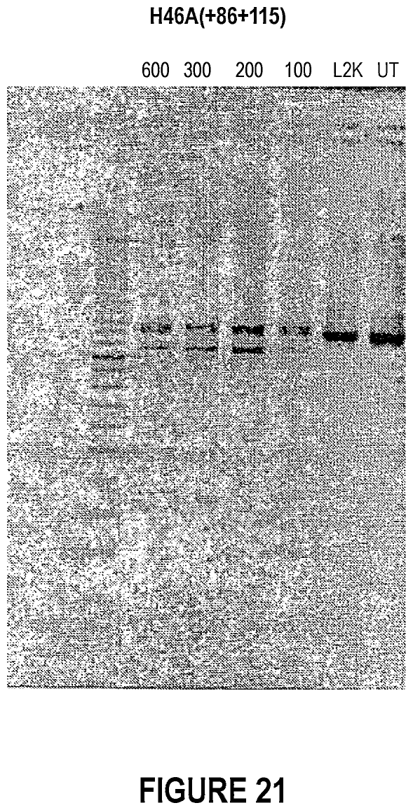

FIG. 21 illustrates the efficiency of one antisense molecule directed at exon 46 acceptor splice site. Antisense oligonucleotide H46A(+86+115) [SEQ ID NO: 203] showed very strong ability to induce exon 46 skipping. Table 36 below includes antisense molecules tested. These antisense molecules showed varying ability to induce exon 46 skipping.

TABLE 36

| | Antisense | | |
|---|---|---|---|
| | oligonucleotide | | Ability to |
| SEQ ID | name | Sequence | induce skipping |
| 168 | H46D(+16 −04) | UUA CCU UGA CUU GCU CAA GC | No skipping |
| 169 | H46A(+90 +109) | UCC AGG UUC AAG UGG GAU AC | No skipping |
| 203 | H46A(+86 +115) | CUC UUU UCC AGG UUC AAG UGG | Good skipping |
| | | GAU ACU AGC | to 100 nM |
| 204 | H46A(+107 +137) | CAA GCU UUU CUU UUA GUU GCU | Good skipping |
| | | GCU CUU UUC C | to 100 nM |
| 205 | H46A(−10 +20) | UAU UCU UUU GUU CUU CUA GCC | Weak skipping |
| | | UGG AGA AAG | |
| 206 | H46A(+50 +77) | CUG CUU CCU CCA ACC AUA AAA | Weak skipping |
| | | CAA AUU C | |

### Antisense Oligonucleotides Directed at Exon 47

Antisense oligonucleotides directed at exon 47 were prepared and tested for their ability to induce exon skipping in human muscle cells using similar methods as described above.

H47A(+76+100) [SEQ ID NO:170] and H47A(−09+12) [SEQ ID NO:172] both induced exon 47 skipping when delivered into cells at a concentration of 200 nM. H47D(+25−02) [SEQ ID NO: 171] is yet to be prepared and tested.

### Antisense Oligonucleotides Directed at Exon 50

Antisense oligonucleotides directed at exon 50 were prepared and tested for their ability to induce exon skipping in human muscle cells using similar methods as described above.

Antisense oligonucleotide molecule H50A(+02+30) [SEQ ID NO: 173] was a strong inducer of exon skipping. Further, H50A(+07+33) [SEQ ID NO:174] and H50D(+07−18) [SEQ ID NO:175] both induced exon 50 skipping when delivered into cells at a concentration of 100 nM.

### Antisense Oligonucleotides Directed at Exon 51

Antisense oligonucleotides directed at exon 51 were prepared and tested for their ability to induce exon skipping in human muscle cells using similar methods as described above.

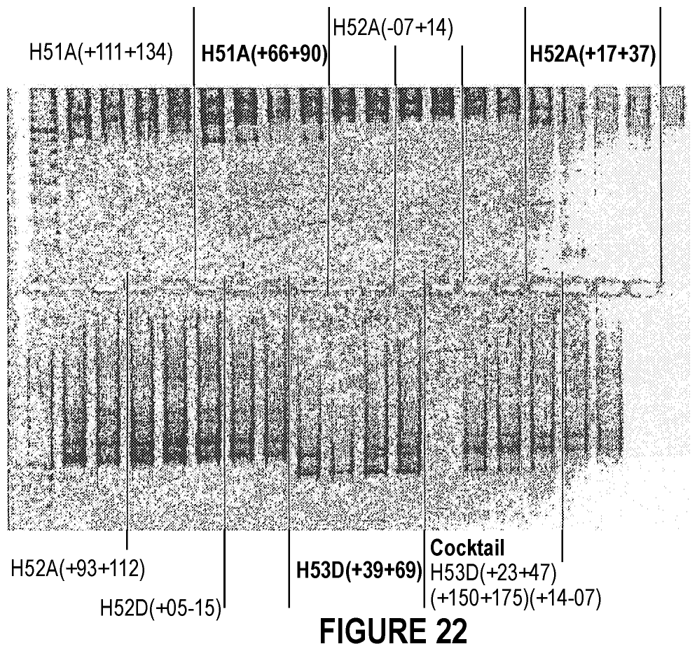

FIG. 22 illustrates differing efficiencies of two antisense molecules directed at exon 51 acceptor splice site. Antisense oligonucleotide H51A(+66+90) [SEQ ID NO: 180] showed the stronger ability to induce exon 51 skipping. Table 37 below includes antisense molecules tested at a concentration range of 25, 50, 100, 300 and 600 nM. These antisense molecules showed varying ability to induce exon 51 skipping. The strongest inducers of exon skipping were antisense oligonucleotide H51A(+61+90) [SEQ ID NO: 179] and H51A(+66+95) [SEQ ID NO: 181].

TABLE 37

| | Antisense | | |
|---|---|---|---|
| | oligonucleotide | | Ability to |
| SEQ ID | name | Sequence | induce skipping |
| 176 | H51A(−01 +25) | ACC AGA GUA ACA GUC | Faint skipping |
| | | UGA GUA GGA GC | |
| 177 | H51D(+16 −07) | CUC AUA CCU UCU GCU | Skipping at 300 nM |
| | | UGA UGA UC | |
| 178 | H51A(+111 +134) | UUC UGU CCA AGC CCG | Needs re-testing |
| | | GUU GAA AUC | |
| 179 | H51A(+61 +90) | ACA UCA AGG AAG AUG | Very strong |
| | | GCA UUU CUA GUU UGG | skipping |
| 180 | H51A(+66 +90) | ACA UCA AGG AAG AUG | skipping |
| | | GCA UUU CUA G | |
| 181 | H51A(+66 +95) | CUC CAA CAU CAA GGA | Very strong |
| | | AGA UGG CAU UUC UAG | skipping |
| 182 | H51D(+08 −17) | AUC AUU UUU UCU CAU | No skipping |
| | | ACC UUC UGC U | |
| 183 | H51A/D(+08 −17) & | AUC AUU UUU UCU CAU | No skipping |
| | (−15 +?) | ACC UUC UGC UAG GAG | |
| | | CUA AAA | |
| 184 | H51A(+175 +195) | CAC CCA CCA UCA GCC | No skipping |
| | | UCU GUG | |
| 185 | H51A(+199 +220) | AUC AUC UCG UUG AUA | No skipping |
| | | UCC UCA A | |

### Antisense Oligonucleotides Directed at Exon 52

Antisense oligonucleotides directed at exon 52 were prepared and tested for their ability to induce exon skipping in human muscle cells using similar methods as described above.

FIG. 22 also shows differing efficiencies of four antisense molecules directed at exon 52 acceptor splice site. The most effective antisense oligonucleotide for inducing exon 52 skipping was H52A(+17+37) [SEQ ID NO: 188).

Table 38 below shows antisense molecules tested at a concentration range of 50, 100, 300 and 600 nM. These antisense molecules showed varying ability to induce exon 50 skipping. Antisense molecules H52A(+12+41) [SEQ ID NO: 187] and H52A(+17+37) [SEQ ID NO:188] showed the strongest exon 50 skipping at a concentration of 50 nM.

TABLE 38

| | Antisense | | |
|---|---|---|---|
| | oligonucleotide | | Ability to |
| SEQ ID | name | Sequence | induce skipping |
| 186 | H52A(−07 +14) | UCC UGC AUU GUU GCC UGU AAG | No skipping |
| 187 | H52A(+12 +41) | UCC AAC UGG GGA CGC CUC UGU | Very strong |
| | | UCC AAA UCC | skipping |
| 188 | H52A(+17 +37) | ACU GGG GAC GCC UCU GUU CCA | Skipping to 50 nM |
| 189 | H52A(+93 +112) | CCG UAA UGA UUG UUC UAG CC | No skipping |
| 190 | H52D(+05 −15) | UGU UAA AAA ACU UAC UUC GA | No skipping |

### Antisense Oligonucleotides Directed at Exon 53

Antisense oligonucleotides directed at exon 53 were prepared and tested for their ability to induce exon skipping in human muscle cells using similar methods as described above.

FIG. 22 also shows antisense molecule H53A(+39+69) [SEQ ID NO:193] directed at exon 53 acceptor splice site. This antisense oligonucleotide was able to induce exon 53 skipping at 5, 100, 300 and 600 nM. A “cocktail” of three exon 53 antisense oligonucleotides: H53A(+23+47) [SEQ ID NO:195], H53A(+150+176) [SEQ ID NO:196] and H53D(+14−07) [SEQ ID NO:194], was also tested, as shown in FIG. 20 and exhibited an ability to induce exon skipping.

Table 39 below includes other antisense molecules tested at a concentration range of 50, 100, 300 and 600 nM. These antisense molecules showed varying ability to induce exon 53 skipping. Antisense molecule H53A(+39+69) [SEQ ID NO:193] induced the strongest exon 53 skipping.

TABLE 39

| | Antisense | | |
|---|---|---|---|
| | oligonucleotide | | Ability to |
| SEQ ID | name | Sequence | induce skipping |
| 191 | H53A(+45 +69) | CAU UCA ACU GUU GCC UCC | Faint skipping |
| | | GGU UCU G | at 50 nM |
| 192 | H53A(+39 +62) | CUG UUG CCU CCG GUU CUG | Faint skipping |
| | | AAG GUG | at 50 nM |
| 193 | H53A(+39 +69) | CAU UCA ACU GUU GCC UCC | Strong skipping |
| | | GGU UCU GAA GGU G | to 50 nM |
| 194 | H53D(+14 −07) | UAC UAA CCU UGG UUU CUG | Very faint |
| | | UGA | skipping to 50 nM |
| 195 | H53A(+23 +47) | CUG AAG GUG UUC UUG | Very faint |
| | | UAC UUC AUC C | skipping to 50 nM |
| 196 | H53A(+150 +176) | UGU AUA GGG ACC CUC CUU | Very faint |
| | | CCA UGA CUC | skipping to 50 nM |
| 197 | H53D(+20 −05) | CUA ACC UUG GUU UCU GUG | Not made yet |
| | | AUU UUC U | |
| 198 | H53D(+09 −18) | GGU AUC UUU GAU ACU | Faint at 600 nM |
| | | AAC CUU GGU UUC | |
| 199 | H53A(−12 +10) | AUU CUU UCA ACU AGA | No skipping |
| | | AUA AAA G | |
| 200 | H53A(−07 +18) | GAU UCU GAA UUG UUU | No skipping |
| | | CAA CUA GAA U | |
| 201 | H53A(+07 +26) | AUC CCA CUG AUU CUG AAU | No skipping |
| | | UC | |
| 202 | H53A(+124 +145) | UUG GCU CUG GCC UGU CCU | No skipping |
| | | AAG A | |

1. An antisense molecule capable of binding to a selected target site to induce exon skipping in the dystrophin gene, as set forth in SEQ ID NO: 1 to 202.

2. An antisense molecule according to claim 1 capable of inducing exon skipping in exons 3, 4, 8, 10 to 16, 19 to 40, 42 to 44, 46, 47 and 50 to 53 of the dystrophin gene.

3. A combination of two or more antisense molecules according to claim 1 capable of binding to a selected target to induce exon skipping in the dystrophin gene.

4. A combination or two or more antisense molecules according to claim 3 selected from Table 1B.

5. A combination of two or more antisense molecules according to claim 1 joined together to form a “weasel”, wherein said weasel is capable of binding to a selected target to induce exon skipping in the dystrophin gene.

6. A combination of two or more antisense molecules according to claim 5 selected from Table 1C.

7. The antisense molecule according to claim 1, capable of binding to a selected target site, wherein the target site is an mRNA splicing site selected from a splicer donor site, splice acceptor sites or exonic splicing enhancer elements.

8. A method of treating muscular dystrophy in a patient comprising administering to the patient a composition comprising an antisense molecule according to claim 1.

9. A pharmaceutical or therapeutic composition for the treatment of muscular dystrophy in a patient comprising (a) at least an antisense molecule according to claim 1, and (b) one or more pharmaceutically acceptable carriers and/or diluents.

10. The composition according to claim 9, comprising about 20 nM to 600 nM of the antisense molecule.

11. The use of an antisense molecule according to claim 1 for the manufacture of a medicament for modulation of muscular dystrophy.

12. An antisense molecule according to claim 1 for use in antisense molecule based therapy.

13. An antisense molecule according to claim 1 as herein before described with reference to the examples.

14. A kit comprising at least one antisense molecule according to claim 1, a suitable carrier and instructions for its use.


SEQUENCE LISTING (214 sequences)

SEQ ID NO: 1 - RNA, 24 nt, synthetic construct
  source 1..24: mol_type = other RNA; note = Synthetic Human 2'-O-methyl phosphorothioate antisense oligonucleotide
gataggtggtatcaacatctgtaa

SEQ ID NO: 2 - RNA, 21 nt, synthetic construct
  source 1..21: mol_type = other RNA; note = Synthetic Human 2'-O-methyl phosphorothioate antisense oligonucleotide
gataggtggtatcaacatctg

SEQ ID NO: 3 - RNA, 25 nt, synthetic construct
  source 1..25: mol_type = other RNA; note = Synthetic Human 2'-O-methyl phosphorothioate antisense oligonucleotide
gataggtggtatcaacatctgtaag

SEQ ID NO: 4 - RNA, 20 nt, synthetic construct
  source 1..20: mol_type = other RNA; note = Synthetic Human 2'-O-methyl phosphorothioate antisense oligonucleotide
ggtggtatcaacatctgtaa

SEQ ID NO: 5 - RNA, 20 nt, synthetic construct
  source 1..20: mol_type = other RNA; note = Synthetic Human 2'-O-methyl phosphorothioate antisense oligonucleotide
gtatcaacatctgtaagcac

SEQ ID NO: 6 - RNA, 23 nt, synthetic construct
  source 1..23: mol_type = other RNA; note = Synthetic Human 2'-O-methyl phosphorothioate antisense oligonucleotide
tgcatgttccagtcgttgtgtgg

SEQ ID NO: 7 - RNA, 25 nt, synthetic construct
  source 1..25: mol_type = other RNA; note = Synthetic Human 2'-O-methyl phosphorothioate antisense oligonucleotide
cactattccagtcaaataggtctgg

SEQ ID NO: 8 - RNA, 25 nt, synthetic construct
  source 1..25: mol_type = other RNA; note = Synthetic Human 2'-O-methyl phosphorothioate antisense oligonucleotide
atttaccaaccttcaggatcgagta

SEQ ID NO: 9 - RNA, 21 nt, synthetic construct
  source 1..21: mol_type = other RNA; note = Synthetic Human 2'-O-methyl phosphorothioate antisense oligonucleotide
ggcctaaaacacatacacata

SEQ ID NO: 10 - RNA, 20 nt, synthetic construct
  source 1..20: mol_type = other RNA; note = Synthetic Canine 2'-O-methyl phosphorothioate antisense oligonucleotide
catttttgacctacatgtgg

SEQ ID NO: 11 - RNA, 20 nt, synthetic construct
  source 1..20: mol_type = other RNA; note = Synthetic Canine 2'-O-methyl phosphorothioate antisense oligonucleotide
tttgacctacatgtggaaag

SEQ ID NO: 12 - RNA, 26 nt, synthetic construct
  source 1..26: mol_type = other RNA; note = Synthetic Canine 2'-O-methyl phosphorothioate antisense oligonucleotide
tacatttttgacctacatgtggaaag

SEQ ID NO: 13 - RNA, 22 nt, synthetic construct
  source 1..22: mol_type = other RNA; note = Synthetic Canine 2'-O-methyl phosphorothioate antisense oligonucleotide
atttttgacctacatgggaaag

SEQ ID NO: 14 - RNA, 23 nt, synthetic construct
  source 1..23: mol_type = other RNA; note = Synthetic Canine 2'-O-methyl phosphorothioate antisense oligonucleotide
tacgagttgattgtcggacccag

SEQ ID NO: 15 - RNA, 25 nt, synthetic construct
  source 1..25: mol_type = other RNA; note = Synthetic Canine 2'-O-methyl phosphorothioate antisense oligonucleotide
gtggtctccttacctatgactgtgg

SEQ ID NO: 16 - RNA, 17 nt, synthetic construct
  source 1..17: mol_type = other RNA; note = Synthetic Canine 2'-O-methyl phosphorothioate antisense oligonucleotide
ggtctccttacctatga

SEQ ID NO: 17 - RNA, 24 nt, synthetic construct
  source 1..24: mol_type = other RNA; note = Synthetic Human 2'-O-methyl phosphorothioate antisense oligonucleotide
tgtctcagtaatcttcttacctat

SEQ ID NO: 18 - RNA, 24 nt, synthetic construct
  source 1..24: mol_type = other RNA; note = Synthetic Human 2'-O-methyl phosphorothioate antisense oligonucleotide
tcttacctatgactatggatgaga

SEQ ID NO: 19 - RNA, 20 nt, synthetic construct
  source 1..20: mol_type = other RNA; note = Synthetic Human 2'-O-methyl phosphorothioate antisense oligonucleotide
gcatgaactcttgtggatcc

SEQ ID NO: 20 - RNA, 20 nt, synthetic construct
  source 1..20: mol_type = other RNA; note = Synthetic Human 2'-O-methyl phosphorothioate antisense oligonucleotide
ccagggtactacttacatta

SEQ ID NO: 21 - RNA, 21 nt, synthetic construct
  source 1..21: mol_type = other RNA; note = Synthetic Human 2'-O-methyl phosphorothioate antisense oligonucleotide
atcgtgtgtcacagcatccag

SEQ ID NO: 22 - RNA, 30 nt, synthetic construct
  source 1..30: mol_type = other RNA; note = Synthetic Human 2'-O-methyl phosphorothioate antisense oligonucleotide
tgttcagggcatgaactcttgtggatcctt

SEQ ID NO: 23 - RNA, 31 nt, synthetic construct
  source 1..31: mol_type = other RNA; note = Synthetic Human 2'-O-methyl phosphorothioate antisense oligonucleotide
taggaggcgcctcccatcctgtaggtcactg

SEQ ID NO: 24 - RNA, 31 nt, synthetic construct
  source 1..31: mol_type = other RNA; note = Synthetic Human 2'-O-methyl phosphorothioate antisense oligonucleotide
aggtctaggaggcgcctcccatcctgtaggt

SEQ ID NO: 25 - RNA, 25 nt, synthetic construct
  source 1..25: mol_type = other RNA; note = Synthetic Human 2'-O-methyl phosphorothioate antisense oligonucleotide
gcgcctcccatcctgtaggtcactg

SEQ ID NO: 26 - RNA, 26 nt, synthetic construct
  source 1..26: mol_type = other RNA; note = Synthetic Human 2'-O-methyl phosphorothioate antisense oligonucleotide
cttcgaggaggtctaggaggcgcctc

SEQ ID NO: 27 - RNA, 21 nt, synthetic construct
  source 1..21: mol_type = other RNA; note = Synthetic Human 2'-O-methyl phosphorothioate antisense oligonucleotide
ctcccatcctgtaggtcactg

SEQ ID NO: 28 - RNA, 22 nt, synthetic construct
  source 1..22: mol_type = other RNA; note = Synthetic Human 2'-O-methyl phosphorothioate antisense oligonucleotide
taccagtttttgccctgtcagg

SEQ ID NO: 29 - RNA, 26 nt, synthetic construct
  source 1..26: mol_type = other RNA; note = Synthetic Human 2'-O-methyl phosphorothioate antisense oligonucleotide
tcaatatgctgcttcccaaactgaaa

SEQ ID NO: 30 - RNA, 25 nt, synthetic construct
  source 1..25: mol_type = other RNA; note = Synthetic Human 2'-O-methyl phosphorothioate antisense oligonucleotide
ctaggaggcgcctcccatcctgtag

SEQ ID NO: 31 - RNA, 31 nt, synthetic construct
  source 1..31: mol_type = other RNA; note = Synthetic Human 2'-O-methyl phosphorothioate antisense oligonucleotide
ttatgatttccatctacgatgtcagtacttc

SEQ ID NO: 32 - RNA, 31 nt, synthetic construct
  source 1..31: mol_type = other RNA; note = Synthetic Human 2'-O-methyl phosphorothioate antisense oligonucleotide
cttacctgccagtggaggattatattccaaa

SEQ ID NO: 33 - RNA, 25 nt, synthetic construct
  source 1..25: mol_type = other RNA; note = Synthetic Human 2'-O-methyl phosphorothioate antisense oligonucleotide
catcaggattcttacctgccagtgg

SEQ ID NO: 34 - RNA, 25 nt, synthetic construct
  source 1..25: mol_type = other RNA; note = Synthetic Human 2'-O-methyl phosphorothioate antisense oligonucleotide
cgatgtcagtacttccaatattcac

SEQ ID NO: 35 - RNA, 18 nt, synthetic construct
  source 1..18: mol_type = other RNA; note = Synthetic Human 2'-O-methyl phosphorothioate antisense oligonucleotide
accattcatcaggattct

SEQ ID NO: 36 - RNA, 18 nt, synthetic construct
  source 1..18: mol_type = other RNA; note = Synthetic Human 2'-O-methyl phosphorothioate antisense oligonucleotide
acctgccagtggaggatt

SEQ ID NO: 37 - RNA, 27 nt, synthetic construct
  source 1..27: mol_type = other RNA; note = Synthetic Human 2'-O-methyl phosphorothioate antisense oligonucleotide
ccaatattcactaaatcaacctgttaa

SEQ ID NO: 38 - RNA, 30 nt, synthetic construct
  source 1..30: mol_type = other RNA; note = Synthetic Human 2'-O-methyl phosphorothioate antisense oligonucleotide
caggattcttacctgccagtggaggattat

SEQ ID NO: 39 - RNA, 31 nt, synthetic construct
  source 1..31: mol_type = other RNA; note = Synthetic Human 2'-O-methyl phosphorothioate antisense oligonucleotide
acgatgtcagtacttccaatattcactaaat

SEQ ID NO: 40 - RNA, 31 nt, synthetic construct
  source 1..31: mol_type = other RNA; note = Synthetic Human 2'-O-methyl phosphorothioate antisense oligonucleotide
atttccatctacgatgtcagtacttccaata

SEQ ID NO: 41 - RNA, 21 nt, synthetic construct
  source 1..21: mol_type = other RNA; note = Synthetic Human 2'-O-methyl phosphorothioate antisense oligonucleotide
caggagcttccaaatgctgca

SEQ ID NO: 42 - RNA, 29 nt, synthetic construct
  source 1..29: mol_type = other RNA; note = Synthetic Human 2'-O-methyl phosphorothioate antisense oligonucleotide
cttgtcttcaggagcttccaaatgctgca

SEQ ID NO: 43 - RNA, 22 nt, synthetic construct
  source 1..22: mol_type = other RNA; note = Synthetic Human 2'-O-methyl phosphorothioate antisense oligonucleotide
tcctcagcagaaagaagccacg

SEQ ID NO: 44 - RNA, 20 nt, synthetic construct
  source 1..20: mol_type = other RNA; note = Synthetic Human 2'-O-methyl phosphorothioate antisense oligonucleotide
ttagaaatctctccttgtgc

SEQ ID NO: 45 - RNA, 20 nt, synthetic construct
  source 1..20: mol_type = other RNA; note = Synthetic Human 2'-O-methyl phosphorothioate antisense oligonucleotide
taaattgggtgttacacaat

SEQ ID NO: 46 - RNA, 24 nt, synthetic construct
  source 1..24: mol_type = other RNA; note = Synthetic Human 2'-O-methyl phosphorothioate antisense oligonucleotide
ccctgaggcattcccatcttgaat

SEQ ID NO: 47 - RNA, 20 nt, synthetic construct
  source 1..20: mol_type = other RNA; note = Synthetic Human 2'-O-methyl phosphorothioate antisense oligonucleotide
aggacttacttgctttgttt

SEQ ID NO: 48 - RNA, 23 nt, synthetic construct
  source 1..23: mol_type = other RNA; note = Synthetic Human 2'-O-methyl phosphorothioate antisense oligonucleotide
cttgaatttaggagattcatctg

SEQ ID NO: 49 - RNA, 23 nt, synthetic construct
  source 1..23: mol_type = other RNA; note = Synthetic Human 2'-O-methyl phosphorothioate antisense oligonucleotide
catcttctgataattttcctgtt

SEQ ID NO: 50 - RNA, 24 nt, synthetic construct
  source 1..24: mol_type = other RNA; note = Synthetic Human 2'-O-methyl phosphorothioate antisense oligonucleotide
tcttctgtttttgttagccagtca

SEQ ID NO: 51 - RNA, 20 nt, synthetic construct
  source 1..20: mol_type = other RNA; note = Synthetic Human 2'-O-methyl phosphorothioate antisense oligonucleotide
tctatgtaaactgaaaattt

SEQ ID NO: 52 - RNA, 20 nt, synthetic construct
  source 1..20: mol_type = other RNA; note = Synthetic Human 2'-O-methyl phosphorothioate antisense oligonucleotide
ttctggagatccattaaaac

SEQ ID NO: 53 - RNA, 24 nt, synthetic construct
  source 1..24: mol_type = other RNA; note = Synthetic Human 2'-O-methyl phosphorothioate antisense oligonucleotide
cagcagttgcgtgatctccactag

SEQ ID NO: 54 - RNA, 21 nt, synthetic construct
  source 1..21: mol_type = other RNA; note = Synthetic Human 2'-O-methyl phosphorothioate antisense oligonucleotide
ttcatcaactaccaccaccat

SEQ ID NO: 55 - RNA, 25 nt, synthetic construct
  source 1..25: mol_type = other RNA; note = Synthetic Human 2'-O-methyl phosphorothioate antisense oligonucleotide
ctaagcaaaataatctgaccttaag

SEQ ID NO: 56 - RNA, 28 nt, synthetic construct
  source 1..28: mol_type = other RNA; note = Synthetic Human 2'-O-methyl phosphorothioate antisense oligonucleotide
cttgtaaaagaacccagcggtcttctgt

SEQ ID NO: 57 - RNA, 22 nt, synthetic construct
  source 1..22: mol_type = other RNA; note = Synthetic Human 2'-O-methyl phosphorothioate antisense oligonucleotide
catctacagatgtttgcccatc

SEQ ID NO: 58 - RNA, 23 nt, synthetic construct
  source 1..23: mol_type = other RNA; note = Synthetic Human 2'-O-methyl phosphorothioate antisense oligonucleotide
gaaggatgtcttgtaaaagaacc

SEQ ID NO: 59 - RNA, 20 nt, synthetic construct
  source 1..20: mol_type = other RNA; note = Synthetic Human 2'-O-methyl phosphorothioate antisense oligonucleotide
acctgttcttcagtaagacg

SEQ ID NO: 60 - RNA, 24 nt, synthetic construct
  source 1..24: mol_type = other RNA; note = Synthetic Human 2'-O-methyl phosphorothioate antisense oligonucleotide
catgacacacctgttcttcagtaa

SEQ ID NO: 61 - RNA, 20 nt, synthetic construct
  source 1..20: mol_type = other RNA; note = Synthetic Human 2'-O-methyl phosphorothioate antisense oligonucleotide
catttgagaaggatgtcttg

SEQ ID NO: 62 - RNA, 24 nt, synthetic construct
  source 1..24: mol_type = other RNA; note = Synthetic Human 2'-O-methyl phosphorothioate antisense oligonucleotide
atctcccaatacctggagaagaga

SEQ ID NO: 63 - RNA, 31 nt, synthetic construct
  source 1..31: mol_type = other RNA; note = Synthetic Human 2'-O-methyl phosphorothioate antisense oligonucleotide
gccatgcactaaaaaggcactgcaagacatt

SEQ ID NO: 64 - RNA, 24 nt, synthetic construct
  source 1..24: mol_type = other RNA; note = Synthetic Human 2'-O-methyl phosphorothioate antisense oligonucleotide
tctttaaagccagttgtgtgaatc

SEQ ID NO: 65 - RNA, 21 nt, synthetic construct
  source 1..21: mol_type = other RNA; note = Synthetic Human 2'-O-methyl phosphorothioate antisense oligonucleotide
tttctgaaagccatgcactaa

SEQ ID NO: 66 - RNA, 25 nt, synthetic construct
  source 1..25: mol_type = other RNA; note = Synthetic Human 2'-O-methyl phosphorothioate antisense oligonucleotide
gtacatacggccagtttttgaagac

SEQ ID NO: 67 - RNA, 31 nt, synthetic construct
  source 1..31: mol_type = other RNA; note = Synthetic Human 2'-O-methyl phosphorothioate antisense oligonucleotide
ctagatccgcttttaaaacctgttaaaacaa

SEQ ID NO: 68 - RNA, 31 nt, synthetic construct
  source 1..31: mol_type = other RNA; note = Synthetic Human 2'-O-methyl phosphorothioate antisense oligonucleotide
tcttttctagatccgcttttaaaacctgtta

SEQ ID NO: 69 - RNA, 25 nt, synthetic construct
  source 1..25: mol_type = other RNA; note = Synthetic Human 2'-O-methyl phosphorothioate antisense oligonucleotide
ctagatccgcttttaaaacctgtta

SEQ ID NO: 70 - RNA, 23 nt, synthetic construct
  source 1..23: mol_type = other RNA; note = Synthetic Human 2'-O-methyl phosphorothioate antisense oligonucleotide
ccgtcttctgggtcactgactta

SEQ ID NO: 71 - RNA, 26 nt, synthetic construct
  source 1..26: mol_type = other RNA; note = Synthetic Human 2'-O-methyl phosphorothioate antisense oligonucleotide
ctagatccgcttttaaaacctgttaa

SEQ ID NO: 72 - RNA, 20 nt, synthetic construct
  source 1..20: mol_type = other RNA; note = Synthetic Human 2'-O-methyl phosphorothioate antisense oligonucleotide
ccgcttttaaaacctgttaa

SEQ ID NO: 73 - RNA, 26 nt, synthetic construct
  source 1..26: mol_type = other RNA; note = Synthetic Human 2'-O-methyl phosphorothioate antisense oligonucleotide
tggattgctttttcttttctagatcc

SEQ ID NO: 74 - RNA, 25 nt, synthetic construct
  source 1..25: mol_type = other RNA; note = Synthetic Human 2'-O-methyl phosphorothioate antisense oligonucleotide
catgcttccgtcttctgggtcactg

SEQ ID NO: 75 - RNA, 23 nt, synthetic construct
  source 1..23: mol_type = other RNA; note = Synthetic Human 2'-O-methyl phosphorothioate antisense oligonucleotide
gatcttgtttgagtgaatacagt

SEQ ID NO: 76 - RNA, 22 nt, synthetic construct
  source 1..22: mol_type = other RNA; note = Synthetic Human 2'-O-methyl phosphorothioate antisense oligonucleotide
gttatccagccatgcttccgtc

SEQ ID NO: 77 - RNA, 25 nt, synthetic construct
  source 1..25: mol_type = other RNA; note = Synthetic Human 2'-O-methyl phosphorothioate antisense oligonucleotide
tgataattggtatcactaacctgtg

SEQ ID NO: 78 - RNA, 22 nt, synthetic construct
  source 1..22: mol_type = other RNA; note = Synthetic Human 2'-O-methyl phosphorothioate antisense oligonucleotide
gtatcactaacctgtgctgtac

SEQ ID NO: 79 - RNA, 19 nt, synthetic construct
  source 1..19: mol_type = other RNA; note = Synthetic Human 2'-O-methyl phosphorothioate antisense oligonucleotide
ctgctggcatcttgcagtt

SEQ ID NO: 80 - RNA, 31 nt, synthetic construct
  source 1..31: mol_type = other RNA; note = Synthetic Human 2'-O-methyl phosphorothioate antisense oligonucleotide
gcctgagctgatctgctggcatcttgcagtt

SEQ ID NO: 81 - RNA, 28 nt, synthetic construct
  source 1..28: mol_type = other RNA; note = Synthetic Human 2'-O-methyl phosphorothioate antisense oligonucleotide
ctggcagaattcgatccaccggctgttc

SEQ ID NO: 82 - RNA, 22 nt, synthetic construct
  source 1..22: mol_type = other RNA; note = Synthetic Human 2'-O-methyl phosphorothioate antisense oligonucleotide
cagcagtagttgtcatctgctc

SEQ ID NO: 83 - RNA, 19 nt, synthetic construct
  source 1..19: mol_type = other RNA; note = Synthetic Human 2'-O-methyl phosphorothioate antisense oligonucleotide
tgatggggtggtgggttgg

SEQ ID NO: 84 - RNA, 25 nt, synthetic construct
  source 1..25: mol_type = other RNA; note = Synthetic Human 2'-O-methyl phosphorothioate antisense oligonucleotide
atctgcattaacaccctctagaaag

SEQ ID NO: 85 - RNA, 24 nt, synthetic construct
  source 1..24: mol_type = other RNA; note = Synthetic Human 2'-O-methyl phosphorothioate antisense oligonucleotide
ccggctgttcagttgttctgaggc

SEQ ID NO: 86 - RNA, 28 nt, synthetic construct
  source 1..28: mol_type = other RNA; note = Synthetic Human 2'-O-methyl phosphorothioate antisense oligonucleotide
atctgcattaacaccctctagaaagaaa

SEQ ID NO: 87 - RNA, 28 nt, synthetic construct
  source 1..28: mol_type = other RNA; note = Synthetic Human 2'-O-methyl phosphorothioate antisense oligonucleotide
gaaggagaagagattcttaccttacaaa

SEQ ID NO: 88 - RNA, 20 nt, synthetic construct
  source 1..20: mol_type = other RNA; note = Synthetic Human 2'-O-methyl phosphorothioate antisense oligonucleotide
attcgatccaccggctgttc

SEQ ID NO: 89 - RNA, 20 nt, synthetic construct
  source 1..20: mol_type = other RNA; note = Synthetic Human 2'-O-methyl phosphorothioate antisense oligonucleotide
cagcagtagttgtcatctgc

SEQ ID NO: 90 - RNA, 22 nt, synthetic construct
  source 1..22: mol_type = other RNA; note = Synthetic Human 2'-O-methyl phosphorothioate antisense oligonucleotide
gccggttgacttcatcctgtgc

SEQ ID NO: 91 - RNA, 22 nt, synthetic construct
  source 1..22: mol_type = other RNA; note = Synthetic Human 2'-O-methyl phosphorothioate antisense oligonucleotide
ctgcatccaggaacatgggtcc

SEQ ID NO: 92 - RNA, 23 nt, synthetic construct
  source 1..23: mol_type = other RNA; note = Synthetic Human 2'-O-methyl phosphorothioate antisense oligonucleotide
gtctgcatccaggaacatgggtc

SEQ ID NO: 93 - RNA, 24 nt, synthetic construct
  source 1..24: mol_type = other RNA; note = Synthetic Human 2'-O-methyl phosphorothioate antisense oligonucleotide
gttgaagatctgatagccggttga

SEQ ID NO: 94 - RNA, 24 nt, synthetic construct
  source 1..24: mol_type = other RNA; note = Synthetic Human 2'-O-methyl phosphorothioate antisense oligonucleotide
tacttactgtctgtagctctttct

SEQ ID NO: 95 - RNA, 24 nt, synthetic construct
  source 1..24: mol_type = other RNA; note = Synthetic Human 2'-O-methyl phosphorothioate antisense oligonucleotide
cactcatggtctcctgatagcgca

SEQ ID NO: 96 - RNA, 22 nt, synthetic construct
  source 1..22: mol_type = other RNA; note = Synthetic Human 2'-O-methyl phosphorothioate antisense oligonucleotide
ctgcaattccccgagtctctgc

SEQ ID NO: 97 - RNA, 23 nt, synthetic construct
  source 1..23: mol_type = other RNA; note = Synthetic Human 2'-O-methyl phosphorothioate antisense oligonucleotide
actgctggacccatgtcctgatg

SEQ ID NO: 98 - RNA, 21 nt, synthetic construct
  source 1..21: mol_type = other RNA; note = Synthetic Human 2'-O-methyl phosphorothioate antisense oligonucleotide
ctaagttgaggtatggagagt

SEQ ID NO: 99 - RNA, 23 nt, synthetic construct
  source 1..23: mol_type = other RNA; note = Synthetic Human 2'-O-methyl phosphorothioate antisense oligonucleotide
tattcacagacctgcaattcccc

SEQ ID NO: 100 - RNA, 26 nt, synthetic construct
  source 1..26: mol_type = other RNA; note = Synthetic Human 2'-O-methyl phosphorothioate antisense oligonucleotide
acagtggtgctgagatagtataggcc

SEQ ID NO: 101 - RNA, 22 nt, synthetic construct
  source 1..22: mol_type = other RNA; note = Synthetic Human 2'-O-methyl phosphorothioate antisense oligonucleotide
taggccactttgttgctcttgc

SEQ ID NO: 102 - RNA, 19 nt, synthetic construct
  source 1..19: mol_type = other RNA; note = Synthetic Human 2'-O-methyl phosphorothioate antisense oligonucleotide
ttcagagggcgctttcttc

SEQ ID NO: 103 - RNA, 23 nt, synthetic construct
  source 1..23: mol_type = other RNA; note = Synthetic Human 2'-O-methyl phosphorothioate antisense oligonucleotide
gggcaggccattcctccttcaga

SEQ ID NO: 104 - RNA, 24 nt, synthetic construct
  source 1..24: mol_type = other RNA; note = Synthetic Human 2'-O-methyl phosphorothioate antisense oligonucleotide
tcttcagggtttgtatgtgattct

SEQ ID NO: 105 - RNA, 27 nt, synthetic construct
  source 1..27: mol_type = other RNA; note = Synthetic Human 2'-O-methyl phosphorothioate antisense oligonucleotide
ctgggctgaattgtctgaatatcactg

SEQ ID NO: 106 - RNA, 26 nt, synthetic construct
  source 1..26: mol_type = other RNA; note = Synthetic Human 2'-O-methyl phosphorothioate antisense oligonucleotide
ctgttggcacatgtgatcccactgag

SEQ ID NO: 107 - RNA, 24 nt, synthetic construct
  source 1..24: mol_type = other RNA; note = Synthetic Human 2'-O-methyl phosphorothioate antisense oligonucleotide
gtctatacctgttggcacatgtga

SEQ ID NO: 108 - RNA, 25 nt, synthetic construct
  source 1..25: mol_type = other RNA; note = Synthetic Human 2'-O-methyl phosphorothioate antisense oligonucleotide
tgctttctgtaattcatctggagtt

SEQ ID NO: 109 - RNA, 26 nt, synthetic construct
  source 1..26: mol_type = other RNA; note = Synthetic Human 2'-O-methyl phosphorothioate antisense oligonucleotide
cctcctttctggcatagaccttccac

SEQ ID NO: 110 - RNA, 25 nt, synthetic construct
  source 1..25: mol_type = other RNA; note = Synthetic Human 2'-O-methyl phosphorothioate antisense oligonucleotide
tgtgtcatccattcgtgcatctctg

SEQ ID NO: 111 - RNA, 25 nt, synthetic construct
  source 1..25: mol_type = other RNA; note = Synthetic Human 2'-O-methyl phosphorothioate antisense oligonucleotide
ttaaggcctcttgtgctacaggtgg

SEQ ID NO: 112 - RNA, 23 nt, synthetic construct
  source 1..23: mol_type = other RNA; note = Synthetic Human 2'-O-methyl phosphorothioate antisense oligonucleotide
gggcctcttctttagctctctga

SEQ ID NO: 113 - RNA, 22 nt, synthetic construct
  source 1..22: mol_type = other RNA; note = Synthetic Human 2'-O-methyl phosphorothioate antisense oligonucleotide
gacttccaaagtcttgcatttc

SEQ ID NO: 114 - RNA, 24 nt, synthetic construct
  source 1..24: mol_type = other RNA; note = Synthetic Human 2'-O-methyl phosphorothioate antisense oligonucleotide
gccaacatgcccaaacttcctaag

SEQ ID NO: 115 - RNA, 26 nt, synthetic construct
  source 1..26: mol_type = other RNA; note = Synthetic Human 2'-O-methyl phosphorothioate antisense oligonucleotide
cagagatttcctcagctccgccagga

SEQ ID NO: 116 - RNA, 21 nt, synthetic construct
  source 1..21: mol_type = other RNA; note = Synthetic Human 2'-O-methyl phosphorothioate antisense oligonucleotide
cttacatctagcacctcagag

SEQ ID NO: 117 - RNA, 25 nt, synthetic construct
  source 1..25: mol_type = other RNA; note = Synthetic Human 2'-O-methyl phosphorothioate antisense oligonucleotide
tccgccatctgttagggtctgtgcc

SEQ ID NO: 118 - RNA, 25 nt, synthetic construct
  source 1..25: mol_type = other RNA; note = Synthetic Human 2'-O-methyl phosphorothioate antisense oligonucleotide
atttgggttatcctctgaatgtcgc

SEQ ID NO: 119 - RNA, 22 nt, synthetic construct
  source 1..22: mol_type = other RNA; note = Synthetic Human 2'-O-methyl phosphorothioate antisense oligonucleotide
catacctcttcatgtagttccc

SEQ ID NO: 120 - RNA, 26 nt, synthetic construct
  source 1..26: mol_type = other RNA; note = Synthetic Human 2'-O-methyl phosphorothioate antisense oligonucleotide
catttgagctgcgtccaccttgtctg

SEQ ID NO: 121 - RNA, 26 nt, synthetic construct
  source 1..26: mol_type = other RNA; note = Synthetic Human 2'-O-methyl phosphorothioate antisense oligonucleotide
tcctgggcagactggatgctctgttc

SEQ ID NO: 122 - RNA, 23 nt, synthetic construct
  source 1..23: mol_type = other RNA; note = Synthetic Human 2'-O-methyl phosphorothioate antisense oligonucleotide
ttgcctgggcttcctgaggcatt

SEQ ID NO: 123 - RNA, 24 nt, synthetic construct
  source 1..24: mol_type = other RNA; note = Synthetic Human 2'-O-methyl phosphorothioate antisense oligonucleotide
ttctgaaataacatatacctgtgc

SEQ ID NO: 124 - RNA, 25 nt, synthetic construct
  source 1..25: mol_type = other RNA; note = Synthetic Human 2'-O-methyl phosphorothioate antisense oligonucleotide
tagtttctgaaataacatatacctg

SEQ ID NO: 125 - RNA, 21 nt, synthetic construct
  source 1..21: mol_type = other RNA; note = Synthetic Human 2'-O-methyl phosphorothioate antisense oligonucleotide
gacttgtcaaatcagattgga

SEQ ID NO: 126 - RNA, 24 nt, synthetic construct
  source 1..24: mol_type = other RNA; note = Synthetic Human 2'-O-methyl phosphorothioate antisense oligonucleotide
gtttctgaaataacatatacctgt

SEQ ID NO: 127 - RNA, 20 nt, synthetic construct
  source 1..20: mol_type = other RNA; note = Synthetic Human 2'-O-methyl phosphorothioate antisense oligonucleotide
caccagaaatacataccaca

SEQ ID NO: 128 - RNA, 20 nt, synthetic construct
  source 1..20: mol_type = other RNA; note = Synthetic Human 2'-O-methyl phosphorothioate antisense oligonucleotide
caatgatttagctgtgactg

SEQ ID NO: 129 - RNA, 23 nt, synthetic construct
  source 1..23: mol_type = other RNA; note = Synthetic Human 2'-O-methyl phosphorothioate antisense oligonucleotide
cgaaacttcatggagacatcttg

SEQ ID NO: 130 - RNA, 25 nt, synthetic construct
  source 1..25: mol_type = other RNA; note = Synthetic Human 2'-O-methyl phosphorothioate antisense oligonucleotide
cttgtagacgctgctcaaaattggc

SEQ ID NO: 131 - RNA, 20 nt, synthetic construct
  source 1..20: mol_type = other RNA; note = Synthetic Human 2'-O-methyl phosphorothioate antisense oligonucleotide
catgcacacacctttgctcc

SEQ ID NO: 132 - RNA, 24 nt, synthetic construct
  source 1..24: mol_type = other RNA; note = Synthetic Human 2'-O-methyl phosphorothioate antisense oligonucleotide
tctgtacaatctgacgtccagtct

SEQ ID NO: 133 - RNA, 27 nt, synthetic construct
  source 1..27: mol_type = other RNA; note = Synthetic Human 2'-O-methyl phosphorothioate antisense oligonucleotide
gtctttatcaccatttccacttcagac

SEQ ID NO: 134 - RNA, 25 nt, synthetic construct
  source 1..25: mol_type = other RNA; note = Synthetic Human 2'-O-methyl phosphorothioate antisense oligonucleotide
ccgtctgctttttctgtacaatctg

SEQ ID NO: 135 - RNA, 22 nt, synthetic construct
  source 1..22: mol_type = other RNA; note = Synthetic Human 2'-O-methyl phosphorothioate antisense oligonucleotide
tccatatctgtagctgccagcc

SEQ ID NO: 136 - RNA, 23 nt, synthetic construct
  source 1..23: mol_type = other RNA; note = Synthetic Human 2'-O-methyl phosphorothioate antisense oligonucleotide
ccaggcaacttcagaatccaaat

SEQ ID NO: 137 - RNA, 30 nt, synthetic construct
  source 1..30: mol_type = other RNA; note = Synthetic Human 2'-O-methyl phosphorothioate antisense oligonucleotide
tttctgttacctgaaaagaattataatgaa

SEQ ID NO: 138 - RNA, 25 nt, synthetic construct
  source 1..25: mol_type = other RNA; note = Synthetic Human 2'-O-methyl phosphorothioate antisense oligonucleotide
cattcatttcctttcgcatcttacg

SEQ ID NO: 139 - RNA, 26 nt, synthetic construct
  source 1..26: mol_type = other RNA; note = Synthetic Human 2'-O-methyl phosphorothioate antisense oligonucleotide
tgatctctttgtcaattccatatctg

SEQ ID NO: 140 - RNA, 30 nt, synthetic construct
  source 1..30: mol_type = other RNA; note = Synthetic Human 2'-O-methyl phosphorothioate antisense oligonucleotide
ttcagtgatataggttttacctttccccag

SEQ ID NO: 141 - RNA, 26 nt, synthetic construct
  source 1..26: mol_type = other RNA; note = Synthetic Human 2'-O-methyl phosphorothioate antisense oligonucleotide
ctgtagctgccagccattctgtcaag

SEQ ID NO: 142 - RNA, 21 nt, synthetic construct
  source 1..21: mol_type = other RNA; note = Synthetic Human 2'-O-methyl phosphorothioate antisense oligonucleotide
tcttctgctcgggaggtgaca

SEQ ID NO: 143 - RNA, 20 nt, synthetic construct
  source 1..20: mol_type = other RNA; note = Synthetic Human 2'-O-methyl phosphorothioate antisense oligonucleotide
ccagttactattcagaagac

SEQ ID NO: 144 - RNA, 20 nt, synthetic construct
  source 1..20: mol_type = other RNA; note = Synthetic Human 2'-O-methyl phosphorothioate antisense oligonucleotide
tcttcaggtgcaccttctgt

SEQ ID NO: 145 - RNA, 25 nt, synthetic construct
  source 1..25: mol_type = other RNA; note = Synthetic Human 2'-O-methyl phosphorothioate antisense oligonucleotide
tgtgatgtggtccacattctggtca

SEQ ID NO: 146 - RNA, 20 nt, synthetic construct
  source 1..20: mol_type = other RNA; note = Synthetic Human 2'-O-methyl phosphorothioate antisense oligonucleotide
ccatgtgtttctggtattcc

SEQ ID NO: 147 - RNA, 25 nt, synthetic construct
  source 1..25: mol_type = other RNA; note = Synthetic Human 2'-O-methyl phosphorothioate antisense oligonucleotide
cgtgtagagtccacctttgggcgta

SEQ ID NO: 148 - RNA, 24 nt, synthetic construct
  source 1..24: mol_type = other RNA; note = Synthetic Human 2'-O-methyl phosphorothioate antisense oligonucleotide
tactaatttcctgcagtggtcacc

SEQ ID NO: 149 - RNA, 24 nt, synthetic construct
  source 1..24: mol_type = other RNA; note = Synthetic Human 2'-O-methyl phosphorothioate antisense oligonucleotide
ttctgtgtgaaatggctgcaaatc

SEQ ID NO: 150 - RNA, 20 nt, synthetic construct
  source 1..20: mol_type = other RNA; note = Synthetic Human 2'-O-methyl phosphorothioate antisense oligonucleotide
ccttcaaaggaatggaggcc

SEQ ID NO: 151 - RNA, 25 nt, synthetic construct
  source 1..25: mol_type = other RNA; note = Synthetic Human 2'-O-methyl phosphorothioate antisense oligonucleotide
tgctgaatttcagcctccagtggtt

SEQ ID NO: 152 - RNA, 25 nt, synthetic construct
  source 1..25: mol_type = other RNA; note = Synthetic Human 2'-O-methyl phosphorothioate antisense oligonucleotide
tgaagtcttcctctttcagattcac

SEQ ID NO: 153 - RNA, 24 nt, synthetic construct
  source 1..24: mol_type = other RNA; note = Synthetic Human 2'-O-methyl phosphorothioate antisense oligonucleotide
ctggctttctctcatctgtgattc

SEQ ID NO: 154 - RNA, 20 nt, synthetic construct
  source 1..20: mol_type = other RNA; note = Synthetic Human 2'-O-methyl phosphorothioate antisense oligonucleotide
gttgtaagttgtctcctctt

SEQ ID NO: 155 - RNA, 20 nt, synthetic construct
  source 1..20: mol_type = other RNA; note = Synthetic Human 2'-O-methyl phosphorothioate antisense oligonucleotide
ttgtctgtaacagctgctgt

SEQ ID NO: 156 - RNA, 20 nt, synthetic construct
  source 1..20: mol_type = other RNA; note = Synthetic Human 2'-O-methyl phosphorothioate antisense oligonucleotide
gctctaataccttgagagca

SEQ ID NO: 157 - RNA, 22 nt, synthetic construct
  source 1..22: mol_type = other RNA; note = Synthetic Human 2'-O-methyl phosphorothioate antisense oligonucleotide
ctttgagacctcaaatcctgtt

SEQ ID NO: 158 - RNA, 25 nt, synthetic construct
  source 1..25: mol_type = other RNA; note = Synthetic Human 2'-O-methyl phosphorothioate antisense oligonucleotide
ctttattttcctttcatctctgggc

SEQ ID NO: 159 - RNA, 27 nt, synthetic construct
  source 1..27: mol_type = other RNA; note = Synthetic Human 2'-O-methyl phosphorothioate antisense oligonucleotide
atcgtttcttcacggacagtgtgctgg

SEQ ID NO: 160 - RNA, 24 nt, synthetic construct
  source 1..24: mol_type = other RNA; note = Synthetic Human 2'-O-methyl phosphorothioate antisense oligonucleotide
gggcttgtgagacatgagtgattt

SEQ ID NO: 161 - RNA, 22 nt, synthetic construct
  source 1..22: mol_type = other RNA; note = Synthetic Human 2'-O-methyl phosphorothioate antisense oligonucleotide
accttcagaggactcctcttgc

SEQ ID NO: 162 - RNA, 25 nt, synthetic construct
  source 1..25: mol_type = other RNA; note = Synthetic Human 2'-O-methyl phosphorothioate antisense oligonucleotide
tatgtgttacctacccttgtcggtc

SEQ ID NO: 163 - RNA, 20 nt, synthetic construct
  source 1..20: mol_type = other RNA; note = Synthetic Human 2'-O-methyl phosphorothioate antisense oligonucleotide
ggagagagcttcctgtagct

SEQ ID NO: 164 - RNA, 23 nt, synthetic construct
  source 1..23: mol_type = other RNA; note = Synthetic Human 2'-O-methyl phosphorothioate antisense oligonucleotide
tcaccctttccacaggcgttgca

SEQ ID NO: 165 - RNA, 20 nt, synthetic construct
  source 1..20: mol_type = other RNA; note = Synthetic Human 2'-O-methyl phosphorothioate antisense oligonucleotide
tttgtgtctttctgagaaac

SEQ ID NO: 166 - RNA, 20 nt, synthetic construct
  source 1..20: mol_type = other RNA; note = Synthetic Human 2'-O-methyl phosphorothioate antisense oligonucleotide
aaagacttaccttaagatac

SEQ ID NO: 167 - RNA, 20 nt, synthetic construct
  source 1..20: mol_type = other RNA; note = Synthetic Human 2'-O-methyl phosphorothioate antisense oligonucleotide
atctgtcaaatcgcctgcag

SEQ ID NO: 168 - RNA, 20 nt, synthetic construct
  source 1..20: mol_type = other RNA; note = Synthetic Human 2'-O-methyl phosphorothioate antisense oligonucleotide
ttaccttgacttgctcaagc

SEQ ID NO: 169 - RNA, 20 nt, synthetic construct
  source 1..20: mol_type = other RNA; note = Synthetic Human 2'-O-methyl phosphorothioate antisense oligonucleotide
tccaggttcaagtgggatac

SEQ ID NO: 170 - RNA, 25 nt, synthetic construct
  source 1..25: mol_type = other RNA; note = Synthetic Human 2'-O-methyl phosphorothioate antisense oligonucleotide
gctcttctgggcttatgggagcact

SEQ ID NO: 171 - RNA, 27 nt, synthetic construct
  source 1..27: mol_type = other RNA; note = Synthetic Human 2'-O-methyl phosphorothioate antisense oligonucleotide
acctttatccactggagatttgtctgc

SEQ ID NO: 172 - RNA, 21 nt, synthetic construct
  source 1..21: mol_type = other RNA; note = Synthetic Human 2'-O-methyl phosphorothioate antisense oligonucleotide
ttccaccagtaactgaaacag

SEQ ID NO: 173 - RNA, 29 nt, synthetic construct
  source 1..29: mol_type = other RNA; note = Synthetic Human 2'-O-methyl phosphorothioate antisense oligonucleotide
ccactcagagctcagatcttctaacttcc

SEQ ID NO: 174 - RNA, 27 nt, synthetic construct
  source 1..27: mol_type = other RNA; note = Synthetic Human 2'-O-methyl phosphorothioate antisense oligonucleotide
cttccactcagagctcagatcttctaa

SEQ ID NO: 175 - RNA, 25 nt, synthetic construct
  source 1..25: mol_type = other RNA; note = Synthetic Human 2'-O-methyl phosphorothioate antisense oligonucleotide
gggatccagtatacttacaggctcc

SEQ ID NO: 176 - RNA, 26 nt, synthetic construct
  source 1..26: mol_type = other RNA; note = Synthetic Human 2'-O-methyl phosphorothioate antisense oligonucleotide
accagagtaacagtctgagtaggagc

SEQ ID NO: 177 - RNA, 23 nt, synthetic construct
  source 1..23: mol_type = other RNA; note = Synthetic Human 2'-O-methyl phosphorothioate antisense oligonucleotide
ctcataccttctgcttgatgatc

SEQ ID NO: 178 - RNA, 24 nt, synthetic construct
  source 1..24: mol_type = other RNA; note = Synthetic Human 2'-O-methyl phosphorothioate antisense oligonucleotide
ttctgtccaagcccggttgaaatc

SEQ ID NO: 179 - RNA, 30 nt, synthetic construct
  source 1..30: mol_type = other RNA; note = Synthetic Human 2'-O-methyl phosphorothioate antisense oligonucleotide
acatcaaggaagatggcatttctagtttgg

SEQ ID NO: 180 - RNA, 25 nt, synthetic construct
  source 1..25: mol_type = other RNA; note = Synthetic Human 2'-O-methyl phosphorothioate antisense oligonucleotide
acatcaaggaagatggcatttctag

SEQ ID NO: 181 - RNA, 30 nt, synthetic construct
  source 1..30: mol_type = other RNA; note = Synthetic Human 2'-O-methyl phosphorothioate antisense oligonucleotide
ctccaacatcaaggaagatggcatttctag

SEQ ID NO: 182 - RNA, 25 nt, synthetic construct
  source 1..25: mol_type = other RNA; note = Synthetic Human 2'-O-methyl phosphorothioate antisense oligonucleotide
atcattttttctcataccttctgct

SEQ ID NO: 183 - RNA, 36 nt, synthetic construct
  source 1..36: mol_type = other RNA; note = Synthetic Human 2'-O-methyl phosphorothioate antisense oligonucleotide
atcattttttctcataccttctgctaggagctaaaa

SEQ ID NO: 184 - RNA, 21 nt, synthetic construct
  source 1..21: mol_type = other RNA; note = Synthetic Human 2'-O-methyl phosphorothioate antisense oligonucleotide
cacccaccatcaccctctgtg

SEQ ID NO: 185 - RNA, 22 nt, synthetic construct
  source 1..22: mol_type = other RNA; note = Synthetic Human 2'-O-methyl phosphorothioate antisense oligonucleotide
atcatctcgttgatatcctcaa

SEQ ID NO: 186 - RNA, 21 nt, synthetic construct
  source 1..21: mol_type = other RNA; note = Synthetic Human 2'-O-methyl phosphorothioate antisense oligonucleotide
tcctgcattgttgcctgtaag

SEQ ID NO: 187 - RNA, 30 nt, synthetic construct
  source 1..30: mol_type = other RNA; note = Synthetic Human 2'-O-methyl phosphorothioate antisense oligonucleotide
tccaactggggacgcctctgttccaaatcc

SEQ ID NO: 188 - RNA, 21 nt, synthetic construct
  source 1..21: mol_type = other RNA; note = Synthetic Human 2'-O-methyl phosphorothioate antisense oligonucleotide
actggggacgcctctgttcca

SEQ ID NO: 189 - RNA, 20 nt, synthetic construct
  source 1..20: mol_type = other RNA; note = Synthetic Human 2'-O-methyl phosphorothioate antisense oligonucleotide
ccgtaatgattgttctagcc

SEQ ID NO: 190 - RNA, 20 nt, synthetic construct
  source 1..20: mol_type = other RNA; note = Synthetic Human 2'-O-methyl phosphorothioate antisense oligonucleotide
tgttaaaaaacttacttcga

SEQ ID NO: 191 - RNA, 25 nt, synthetic construct
  source 1..25: mol_type = other RNA; note = Synthetic Human 2'-O-methyl phosphorothioate antisense oligonucleotide
cattcaactgttgcctccggttctg

SEQ ID NO: 192 - RNA, 24 nt, synthetic construct
  source 1..24: mol_type = other RNA; note = Synthetic Human 2'-O-methyl phosphorothioate antisense oligonucleotide
ctgttgcctccggttctgaaggtg

SEQ ID NO: 193 - RNA, 31 nt, synthetic construct
  source 1..31: mol_type = other RNA; note = Synthetic Human 2'-O-methyl phosphorothioate antisense oligonucleotide
cattcaactgttgcctccggttctgaaggtg

SEQ ID NO: 194 - RNA, 21 nt, synthetic construct
  source 1..21: mol_type = other RNA; note = Synthetic Human 2'-O-methyl phosphorothioate antisense oligonucleotide
tactaaccttggtttctgtga

SEQ ID NO: 195 - RNA, 25 nt, synthetic construct
  source 1..25: mol_type = other RNA; note = Synthetic Human 2'-O-methyl phosphorothioate antisense oligonucleotide
ctgaaggtgttcttgtacttcatcc

SEQ ID NO: 196 - RNA, 27 nt, synthetic construct
  source 1..27: mol_type = other RNA; note = Synthetic Human 2'-O-methyl phosphorothioate antisense oligonucleotide
tgtatagggaccctccttccatgactc

SEQ ID NO: 197 - RNA, 25 nt, synthetic construct
  source 1..25: mol_type = other RNA; note = Synthetic Human 2'-O-methyl phosphorothioate antisense oligonucleotide
ctaaccttggtttctgtgattttct

SEQ ID NO: 198 - RNA, 27 nt, synthetic construct
  source 1..27: mol_type = other RNA; note = Synthetic Human 2'-O-methyl phosphorothioate antisense oligonucleotide
ggtatctttgatactaaccttggtttc

SEQ ID NO: 199 - RNA, 22 nt, synthetic construct
  source 1..22: mol_type = other RNA; note = Synthetic Human 2'-O-methyl phosphorothioate antisense oligonucleotide
attctttcaactagaataaaag

SEQ ID NO: 200 - RNA, 25 nt, synthetic construct
  source 1..25: mol_type = other RNA; note = Synthetic Human 2'-O-methyl phosphorothioate antisense oligonucleotide
gattctgaattctttcaactagaat

SEQ ID NO: 201 - RNA, 20 nt, synthetic construct
  source 1..20: mol_type = other RNA; note = Synthetic Human 2'-O-methyl phosphorothioate antisense oligonucleotide
atcccactgattctgaattc

SEQ ID NO: 202 - RNA, 22 nt, synthetic construct
  source 1..22: mol_type = other RNA; note = Synthetic Human 2'-O-methyl phosphorothioate antisense oligonucleotide
ttggctctggcctgtcctaaga

SEQ ID NO: 203 - RNA, 30 nt, synthetic construct
  source 1..30: mol_type = other RNA; note = Synthetic Human 2'-O-methyl phosphorothioate antisense oligonucleotide
ctcttttccaggttcaagtgggatactagc

SEQ ID NO: 204 - RNA, 31 nt, synthetic construct
  source 1..31: mol_type = other RNA; note = Synthetic Human 2'-O-methyl phosphorothioate antisense oligonucleotide
caagcttttcttttagttgctgctcttttcc

SEQ ID NO: 205 - RNA, 30 nt, synthetic construct
  source 1..30: mol_type = other RNA; note = Synthetic Human 2'-O-methyl phosphorothioate antisense oligonucleotide
tattcttttgttcttctagcctggagaaag

SEQ ID NO: 206 - RNA, 28 nt, synthetic construct
  source 1..28: mol_type = other RNA; note = Synthetic Human 2'-O-methyl phosphorothioate antisense oligonucleotide
ctgcttcctccaaccataaaacaaattc

SEQ ID NO: 207 - RNA, 26 nt, synthetic construct
  source 1..26: mol_type = other RNA; note = Synthetic Human 2'-O-methyl phosphorothioate antisense oligonucleotide
ccaatgccatcctggagttcctgtaa

SEQ ID NO: 208 - RNA, 20 nt, synthetic construct
  source 1..20: mol_type = other RNA; note = Synthetic Human 2'-O-methyl phosphorothioate antisense oligonucleotide
tcctgtagaatactggcatc

SEQ ID NO: 209 - RNA, 27 nt, synthetic construct
  source 1..27: mol_type = other RNA; note = Synthetic Human 2'-O-methyl phosphorothioate antisense oligonucleotide
tgcagacctcctgccaccgcagattca

SEQ ID NO: 210 - RNA, 20 nt, synthetic construct
  source 1..20: mol_type = other RNA; note = Synthetic Human 2'-O-methyl phosphorothioate antisense oligonucleotide
ctacctcttttttctgtctg

SEQ ID NO: 211 - RNA, 20 nt, synthetic construct
  source 1..20: mol_type = other RNA; note = Synthetic Human 2'-O-methyl phosphorothioate antisense oligonucleotide
tgtttttgaggattgctgaa

SEQ ID NO: 212 - RNA, 84 nt, synthetic construct
  source 1..84: mol_type = other RNA; note = Synthetic Human 2'-O-methyl phosphorothioate antisense oligonucleotide
cagcagtagttgtcatctgctcaactggcagaattcgatccaccggctgttcaagcctgagctgatctgctcgcatcttgcagt

SEQ ID NO: 213 - RNA, 44 nt, Homo sapiens
  source 1..44: mol_type = genomic RNA
tcatgcactgagtgacctctttctcgcaggcgctagctggagca

SEQ ID NO: 214 - RNA, 22 nt, Homo sapiens
  source 1..22: mol_type = genomic RNA
ccgtgcagactgacggtctcat